SEQUENCE LISTING (42 sequences)

SEQ ID NO: 1 - DNA, 4383 bp, Homo sapiens
gtgctgagagtgtcctgcctggtcctctgtgcctggtggggtgggggtgccaggtgtgtccagaggagcccatttggtagtgaggcaggtatggggctagaagcactggtgcccctggccgtgatagtggccatcttcctgctcctggtggacctgatgcaccggcgccaacgctgggctgcacgctacccaccaggccccctgccactgcccgggctgggcaacctgctgcatgtggacttccagaacacaccatactgcttcgaccaggtgagggaggaggtcctggagggcggcagaggtgctgaggctcccctaccagaagcaaacatggatggtgggtgaaaccacaggctggaccagaagccaggctgagaaggggaagcaggtttgggggacgtcctggagaagggcatttatacatggcatgaaggactggattttccaaaggccaaggaagagtagggcaagggcctggaggtggagctggacttggcagtgggcatgcaagcccattgggcaacatatgttatggagtacaaagtcccttctgctgacaccagaaggaaaggccttgggaatggaagatgagttagtcctgagtgccgtttaaatcacgaaatcgaggatgaagggggtgcagtgacccggttcaaaccttttgcactgtgggtcctcgggcctcactgctcaccggcatggaccatcatctgggaatgggatgctaactggggcctctcggcaattttggtgactcttgcaaggtcatacctgggtgacgcatccaaactgagttcctccatcacagaaggtgtgacccccacccccgccccacgatcaggaggctgggtctcctccttccacctgctcactcctggtagccccgggggtcgtccaaggttcaaataggactaggacctgtagtctggggtgatcctggcttgacaagaggccctgaccctccctctgcagttgcggcgccgcttcggggacgtgttcagcctgcagctggcctggacgccggtggtcgtgctcaatgggctggcggccgtgcgcgaggcgctggtgacccacggcgaggacaccgccgaccgcccgcctgtgcccatcacccagatcctgggtttcgggccgcgttcccaaggcaagcagcggtggggacagagacagatttccgtgggacccgggtgggtgatgaccgtagtccgagctgggcagagagggcgcggggtcgtggacatgaaacaggccagcgagtggggacagcgggccaagaaaccacctgcactagggaggtgtgagcatggggacgagggcggggcttgtgacgagtgggcggggccactgccgagacctggcaggagcccaatgggtgaggctggcgcatttcccagctggaatccggtgtcgaagtggggggcggggaccgcacctgtgctgtaagctcagtgtgggtggcgcggggcccgcggggtcttccctgagtgcaaaggcggtcagggtgggcagagacgaggtggggcaaagccctgccccagccaagggagcaaggtggatgcacaaagagtgggccctgtgaccagctggacagagccagggactgcgggagaccagggggagcatagggttggagtgggtggtggatggtggggctaatgccttcatggccacgcgcacgtgcccgtcccacccccaggggtgttcctggcgcgctatgggcccgcgtggcgcgagcagaggcgcttctccgtgtccaccttgcgcaacttgggcctgggcaagaagtcgctggagcagtgggtgaccgaggaggccgcctgcctttgtgccgccttcgccaaccactccggtgggtgatgggcagaagggcacaaagcgggaactgggaaggcgggggacggggaaggcgaccccttacccgcatctcccacccccaggacgcccctttcgccccaacggtctcttggacaaagccgtgagcaacgtgatcgcctccctcacctgcgggcgccgcttcgagtacgacgaccctcgcttcctcaggctgctggacctagctcaggagggactgaaggaggagtcgggctttctgcgcgaggtgcggagcgagagaccgaggagtctctgcagggcgagctcccgagaggtgccggggctggactggggcctcggaagagcaggatttgcatagatgggtttgggaaaggacattccaggagaccccactgtaagaagggcctggaggaggaggggacatctcagacatggtcgtgggagaggtgtgcccgggtcagggggcaccaggagaggccaaggactctgtacctcctatccacgtcagagatttcgattttaggtttctcctctgggcaaggagagagggtggaggctggcacttggggagggacttggtgaggtcagtggtaaggacaggcaggccctgggtctacctggagatggctggggcctgagacttgtccaggtgaacgcagagcacaggagggattgagaccccgttctgtctggtgtaggtgctgaatgctgtccccgtcctcctgcatatcccagcgctggctggcaaggtcctacgcttccaaaaggctttcctgacccagctggatgagctgctaactgagcacaggatgacctgggacccagcccagcccccccgagacctgactgaggccttcctggcagagatggagaaggtgagagtggctgccacggtggggggcaagggtggtgggttgagcgtcccaggaggaatgaggggaggctgggcaaaaggttggaccagtgcatcacccggcgagccgcatctgggctgacaggtgcagaattggaggtcatttgggggctaccccgttctgtcccgagtatgctctcggccctgctcaggccaaggggaaccctgagagcagcttcaatgatgagaacctgcgcatagtggtggctgacctgttctctgccgggatggtgaccacctcgaccacgctggcctggggcctcctgctcatgatcctacatccggatgtgcagcgtgagcccatctgggaaacagtgcaggggccgagggaggaagggtacaggcgggggcccatgaactttgctgggacacccggggctccaagcacaggcttgaccaggatcctgtaagcctgacctcctccaacataggaggcaagaaggagtgtcagggccggaccccctgggtgctgacccattgtggggacgcatgtctgtccaggccgtgtccaacaggagatcgacgacgtgatagggcaggtgcggcgaccagagatgggtgaccaggctcacatgccctacaccactgccgtgattcatgaggtgcagcgctttggggacatcgtccccctgggtgtgacccatatgacatcccgtgacatcgaagtacagggcttccgcatccctaaggtaggcctggcgccctcctcaccccagctcagcaccagcacctggtgatagccccagcatggctactgccaggtgggcccactctaggaaccctggccacctagtcctcaatgccaccacactgactgtccccacttgggtggggggtccagagtataggcagggctggcctgtccatccagagcccccgtctagtggggagacaaaccaggacctgccagaatgttggaggacccaacgcctgcagggagagggggcagtgtgggtgcctctgagaggtgtgactgcgccctgctgtggggtcggagagggtactgtggagcttctcgggcgcaggactagttgacagagtccagctgtgtgccaggcagtgtgtgtcccccgtgtgtttggtggcaggggtcccagcatcctagagtccagtccccactctcaccctgcatctcctgcccagggaacgacactcatcaccaacctgtcatcggtgctgaaggatgaggccgtctgggagaagcccttccgcttccaccccgaacacttcctggatgcccagggccactttgtgaagccggaggccttcctgcctttctcagcaggtgcctgtggggagcccggctccctgtccccttccgtggagtcttgcaggggtatcacccaggagccaggctcactgacgcccctcccctccccacaggccgccgtgcatgcctcggggagcccctggcccgcatggagctcttcctcttcttcacctccctgctgcagcacttcagcttctcggtgcccactggacagccccggcccagccaccatggtgtctttgctttcctggtgagcccatccccctatgagctttgtgctgtgccccgctagaatggggtacctagtccccagcctgctccctagccagaggctctaatgtacaataaagcaatgtggtagttccaa

SEQ ID NO: 2 - protein, 497 aa, Homo sapiens [sequence omitted]

SEQ ID NO: 3 - DNA, 778 bp, Homo sapiens
cggtccccactatgggcctggagctgtacctggacctgctgtcccagccctgccgcgctgtttacatctttgccaagaagaacgacattcccttcgagctgcgcatcgtggatctgattaaagtgtggccatcctgctctacctgacgcgcaaatataaggtccctgactactggtaccctcaggacctgcaggcccgtgcccgtgtggatgagtacctggcatggcagcacacgactctgcggagaagctgcctccgggccttgtggcataaggtgatgttccctgttttcctgggtgagccagtatctccccagacactggcagccaccctggcagagttggatgtgaccctgcagttgctcgaggacaagttcctccagaacaaggccttccttactggtcctcacatctccttagctgacctcgtagccatcacggagctgatgcatcccgtgggtgctggctgccaagtcttcgaaggccgacccaagctggccacatggcggcagcgcgtggaggcagcagtgggggaggacctcttccaggaggcccatgaggtcattctgaaggccaaggacttcccacctgcagaccccaccataaagcagaagctgatgccctgggtgctggccatgatccggtgagctgggaaacctcacccttgcaccgtcctcagcagtccacaaagcattttcatttctaatggcccatgggagccaggcccagaaagcaggaatggcttgcttaagacttgcccaagtcccagagcacctcac

SEQ ID NO: 4 - protein, 122 aa, Homo sapiens
MFPVFLGEPVSPQTLAATLAELDVTLQLLEDKFLQNKAFLTGPHISLADLVAITELMHPVGAGCQVFEGRPKLATWRQRVEAAVGEDLFQEAHEVILKAKDFPPADPTIKQKLMPWVLAMIR

SEQ ID NO: 5 - DNA, 19149 bp, Homo sapiens
gatgggattggggttttcccctcccatgtgctcaagactggcgctaaaagttttgagcttctcaaaagtctagagccaccgtccagggagcaggtagctgctgggctccggggacactttgcgttcgggctgggagcgtgctttccacgacggtgacacgcttccctggattgggtaagctcctgactgaacttgatgagtcctctctgagtcacgggctctcggctccgtgtattttcagctcgggaaaatcgctggggctgggggtggggcagtggggacttagcgagtttgggggtgagtgggatggaagcttggctagagggatcatcataggagttgcattgttgggagacctgggtgtagatgatggggatgttaggaccatccgaactcaaagttgaacgcctaggcagaggagtggagctttggggaaccttgagccggcctaaagcgtacttctttgcacatccacccggtgctgggcgtagggaatccctgaaataaaagatgcacaaagcattgaggtctgagacttttggatctcgaaacattgagaactcatagctgtatattttagagcccatggcatcctagtgaaaactggggctccattccgaaatgatcatttgggggtgatccggggagcccaagctgctaaggtcccacaacttccggacctttgtccttcctggagcgatctttccaggcagcccccggctccgctagatggagaaaatccaattgaaggctgtcagtcgtggaagtgagaagtgctaaaccaggggtttgcccgccaggccgaggaggaccgtcgcaatctgagaggcccggcagccctgttattgtttggctccacatttacatttctgcctcttgcagcagcatttccggtttctttttgccggagcagctcactattcacccgatgagaggggaggagagagagagaaaatgtcctttaggccggttcctcttacttggcagagggaggctgctattctccgcctgcatttctttttctggattacttagttatggcctttgcaaaggcaggggtatttgttttgatgcaaacctcaatccctccccttctttgaatggtgtgccccaccccgcgggtcgcctgcaacctaggcggacgctaccatggcgtgagacagggagggaaagaagtgtgcagaaggcaagcccggaggtattttcaagaatgagtatatctcatcttcccggaggaaaaaaaaaaagaatgggtacgtctgagaatcaaattttgaaagagtgcaatgatgggtcgtttgataatttgtcggaaaaacaatctacctgttatctagctttgggctaggccattccagttccagacgcaggctgaacgtcgtgaagcggaaggggcgggcccgcaggcgtccgtgtggtcctccgtgcagccctccggcccgagccggttcttcctggtaggaggcggaactcgaattcatttctcccgctgccccatctcttagctcgcggttgtttcattccgcagtttcttcccatgcacctgccgcgtaccggccactttgtgccgtacttacgtcatctttttcctaaatcgaggtggcatttacacacagcgccagtgcacacagcaagtgcacaggaagatgagttttggcccctaaccgctccgtgatgcctaccaagtcacagacccttttcatcgtcccagaaacgtttcatcacgtctcttcccagtcgattcccgaccccacctttattttgatctccataaccattttgcctgttggagaacttcatatagaatggaatcaggctgggcgctgtggctcacgcctgcactttgggaggccgaggcgggcggattacttgaggataggagttccagaccagcgtggccaacgtggtgaatccccgtctctactaaaaaatacaaaaattagctgggcgtggtgggtgcctgtaatcccagctattcgggagggtgaggcaggagaatcgcttgaacccgggaggcagaggttgcagtgagccaagatcgtgccactacactccagcctgggcgacaagaacgaaactccgtctcaaaaaaaaggggggaatcatacattatgtgctcatttttgtcgggcttctgtccttcaatgtactgtctgacattcgttcatgttgtatatatcagtattttgctccttttcatttagtatagtccatcgattgtatatccgtccttttgatggccttttgagttgtttcccatttgcggttatgaaataaagctgctataaacattcttgtacaattctttttgtgatcatatgttttcgtgtttcttggagaaatacttaggaggggaattgcgagtttggaagtaaaaagtagctgtattttgaactttttcagaagctctgagttttccagagcggttgtaccattttacactccaactagcaaggtatgggagttattatggttgtgccacagccttccggacattaggtattgtcagtctttctaatgtggtatatccttgtggttgtaatttacagttctctattgactaaggatgttcagcattttttcatgtgcctattggccattcgtattttgtttgtaaagtagctcttcgagtcttttacctgttattttggttttttgtttgtttttattgttcagttgtgggactgctttatacattctggatacaagtcctttatcagatccatgtgtcgtgaatgttttcttctgatctgttgcttgcctatttgtttgctttacagagtttacagtatcttaagaggagtggatttatcttttttatgttcagtatttgccttgtcctgtttaggacatctttttttttttttttaaccccagggtcatgaagatattatcttacattttcttttaggacctttatggttgtaagttttacagtaaggtccttgagccattaattaattcttaaaattaattgtttatggtgtgaggtgtaggagtcagtctctggtatctttcctgtatggaaatccagttattctgtctccacttgttgaaataggcttcctttctctactgaatgcttttaattttaattattttacagttggagtatagggctaccattttagtgctattttctttttttctttgttaatttttgagacagggactcacactgttgcccaggctagagtacaatggcacaatcaaggcttactgcagcctcgaacccctgggctcaagcagtcctctagcagcctcacgagtagctgggattactccaccacacccagctaactattttatttttttgtattgacaggatctcactatgttgcccaggctggtctcaaactgctggcctcaagctttcatcccatctcggcctcccaaagtgctgggattacaggtgtgagccaccatgcctgacctcttagtgctattttctatttatctcctctgttctctgctctctttaaacgttggaggaagaaacagtacccatcttacacaaactcttcagaaaacagaggaacagactgggcgcggtggctcatacctgtaatctcagcactttggtacgctgaggcaggggatcatttgaggtcgggagttcgagaccagcctggccaacacggcgaaaccccatctctactaaaaatacaaaaagtagctaggcgtggtgacacatacctgtaatgccagttactcaggaggctgaggcacaagaatcccttgaacctgggaagcggaggttgcagtgagccgagattgcgccactgcactccagcctgggcaacagagtgagaccctgtctcagaaaaaaaaagaaagaaagaaaaaatagaggaatatttcccaacttgttttcgaagccagcataatcctggtaccaaaaccaaacaaggacattataagaaaagaaaatatagaccaatattcctgttagcatagacatgcaacagctaaccaattttagcaaaccaaacctggtaatatagaaaaaaggataaataggccagtcgcggtggctcacgcctgtaatcccagcactttgggaggctgaggcaggcagatcacttgaggtcaggagtttgagaccagcctgaccaacatggtgaaaccccgtttctaataaaaatacaaaaatcaggctgggcacggtggctcacgcctgtaatcccagcactttgggaggccgaggtgggcagatcacgaggtcaggagttcaagaccagcctgaccaatgtggtgaaacgccatctctactaaaaatacaaaaatcagccggtgtggtggcacctgcctgtaatcccagctactcaggaggctgaggcagaattgcttgaacccgggaggcagaggttgcagtgagccaagatcgtgccactgcactccagcctgggcgacagagcaagacttcatctcaaaaaaaaaaaaaaattagctgggcatggtggtgggcacctgaaatcccagctactcgggagtctgaggcaggagaatcgcttgaacccaggaggcagaagttgcactgagctgggatcacaccattgcactccagcctgggcaacagagtgagactccatctcaaaaaaagaaaaagaaaaaggataaatacattctaaccaaataatgtttatctcatgattgtagctgattcaacattcaaaaattggcctggtgcagtagctcaggcctgtaatcccaacattttaggaggctgaggcaggaagatctcttgagcccaggatttcaagaccagcctgggcaacatagtcagactggtctttactggggggaaaaaaatcagtctgtgtaattcaccacattaacaaagggaaacataaaaaccctatgatcatttcaacagatgtagcaaaagcagttaatgatattcaacacatatgcatgattacaaaccaaccaacctcctagcaaactagggaaaggaaacttaacctagtttgataacagggcgtccacagtcggagttccactagcagcatacataatggtagaaaactcagtgctgccgggcgcggtggctcacgcctgtaatgccagcactttgggaggcctaggcgggcggatcacgaggtcaggagatcgagactgtcctgactagcatgctgaaaccccgtctctactaaaaatacaaaaacaaaaaattagccgggcatggtggcgggcgcctatagtcccagctactcgggaggctgaggcgagagaatggcgtgaacccgggaggcggagcttgcagagcctagatcgtgccactgcactccagcctgggtgacagagtgagacttcgtctcaaaaaaaaaaaaaaaaaaaaaagaaaagaaaactcaacgctttttcctctaagatcaggaactagaaaaggatttgactctcacaacgttgataccatactggaggttttaaccaggcaagaaaaagaaataatgagggccgggtgcggtggctcaggcctgtaatcccagcactttgggaagccgagacgggtggatcacgaggtcaggagatcgagaccatcctggctaacacggtgaaaccctgtctctactaaatatacaaaaaattagccgggcgtagtggcgggcgcctgtagtcccagctactcgggaggctgaggcaggagaatggcgtgaactcagggggcggagcttgcagtgagctgagatcgagccactgcactccagcctgggcgacagagcaagactgtgtctcaaaaaaaaaaaaagaaaaagaaataatgattagtggcccgatgtctcacgcctataatcccagcactttgggaggccgaggtgggcagatcacctgaggtctggagttggagaccagcctgacaaagatggtgaaacctcgtctctattaaaatattaaaaaaatagccaggcgttggccgggtacagtggctcatgcctgtaatcccagcactttgggaggccgaggtgggtggatcacctgaggtcaggagttcaacaccagcctggccaacatggtgaaaccccatctctactaaaaatacaaaaattagccgggcgtagtggcgggcgcctgtaatcccagctacttgggaggcttaggcaggagaatcgcttgaacctgggaggcggaggttgtagtgagccgagattgcaccattgcactccagcctgggtgacaaaagcaaaaactccgtctcaaaaaaaaaagaattagccaggggtagtggtgaacgcctgtagtcccagctactcaggaggcagaggcaggagaatcacttgaacccaggaggcagaggttgcagtgagccgagattgtcccattgcactccagcctaggcgacaagagcaaaattccatgtcaaaaaaaaaaaaaaaaaaggaaagaaaaaaaataacgattagaaaggaagaaataaaacacattcacagccagtatgattctatacatacatgtcctaatggggccaggcgtggtggctcatgcctgtaatcctagcacttttaggaggctgaggcaggtggcttccctgggaccagcctggccaacatggtgaaaccccaactctaataaaaatacaaaaaatcagccaggcgtggtgacgggcacctctaatcccagctactcaggaggctgaggcaggagaattgcttggacctgggaggcagaggttgcagtgagccgagatcgcgctattgcactccagcctgggcaacaagagtgaaactccggcagggtgtggtggcttacgcctgtaatcccagcacttcgggaggctgaggcaggccgatcacctgaggtcaggagtttgagaccaacctaacatggtgaaaccccgtctctactaaaaatacaagaattagctgggtgtagtggtgggcgcctgtaatcccagctacttgggaggctgagacagaagaattgcttgaacccaggaggtggaggttgcagtgagctgagatcatgccattgcacaccacgccgggcaacagagcgagattccgtctcaaaaaaaaaaaaaaagagtgaaactctatctcaaaaaaaaaaaaaagtcctaatggaaaatccataaaaagctaccaaaactaataaataaatatagcagggttgcaggttacagggcaatatagttatccctctatctgtaggggcttggttctgggactcctcacacaccaaacccacagatgtctaagtcccatatataagacggtatagtatttggatttaacctacacatatcctcccatatagtttaaattatctctagattacttacattacccccatacaatgaaaatgctaatgtacatgcaagtatgtatgtaagtacttgtactatattgtttagggaatcactggacatataggccttcaagactgataccagcagccactgttaagattctggtcaggcctgcccctgtttggggtctcagttgatctcattgccttcccacccagccaagggcacctgcatttctcttggctccctggccatttggaaggcctagttcagcctggcacatttgtatcctggcccactgatgctggtacccctgggaaggtcctgctctgaaaaacacggagattttagttgctactgaagatttgagagataaagacagggagacctgtctgtagacctgtgtccctccaagtgggattgagactttgggccccccatttcaggacagcacctcctggcctgttgactgaatagatccctgaaggaggtgtacttgcattaatggagtgggggtgggagcagtaccacagatccgcactaacaatcacacagttctctctagaataataatatagaacaagtgaaatagaacaattgcagaaagagctaacctttgttgagctcttactgtgtgcccagcactttcctcaactctacatttcccataatacacagagtactaggtaggccaggcttggtggctcacgcctgtaatcccagcactttaggaggccaaggggggtggatcacctgaggtcgggagttcaagaccagcctgaccaacatggtgaaaccccgtctctactagaagtacaaaattagccaggtgtggtggcacatgcttgtagtcctagctactcagcaggctgaggcaggagaatcatttgaatccgggaggaggttgcagtaagcggagatagtgccactgtactccagcctgggcaataagagctgagactccgtctcaaaataaaataaaataaaataaaataaaataaaataaaataaaaaaagaaaagagcctgccattaaaggagctgtttggtaggggatgttttgtcagtgcaaacaacagaaaagtgggctgggcacagtggttcatgcctgtaatcccagcactttgggaggccaaggcgggcggatcacctgaagttgggagttcaagaccagcctgaccaatatggagaaaccccgtctctactaaaaatacaaaattagccgggcgcagtggcgcatgcctgtaatcccagctactcgggaggctgaggcaggagaatcgcttgaacctgggaggcagaggttgcggtgagccgagatcgcaccattgcactccagcctggacgagagcaaaactctgtctcaaaaaaaaaaaaaaacagaaaagtgtaacaaacacttacagtaggcatgtttcttagcaaatctgatgacaaatttggcataaagaaagagagcatccctgaaaaaaaaaaaaagaaaaagaaagagagcatcctgcctgggcaacatagtgaaaccctgcctctacaaaaaaactcaaaaattggccgggtgcagtggctcacacctgtaatcccagcactttgggagtcggaggcgggaggatcacctgaggtcaggagttcgaaaccagcctggccaacatggcaaaaccccatctctactaaaaatacaaaaaattaatcaggcgcattggtgggcgcctgtaatcccagctactcaggaagttgaggcaagaggatcgcttgaatctgggaggtggaggttacagtgagtcgagatcacaccactgcactctagcctgggtgacagggcgagactccgtctccaaaaaaaaaaagaaaaagaaaaagactaaaaaattagccaggcaggcctctgtggtcccagctacttgggaggctgaggcaggagaatcactgagcccaggagtccgaggctgtagtgagccatgattgcaccactgtaccctagcttgggcaacaaagcaagaccctgcctcaaaagaaaaaagaaagaaagaaagaacatggcgggccaggcacagtggctcacacctgtaatcccagcgctttgagaggccgaggcaggtggatcacaaggtcaggagttccacaccagcctggccaacatggtgaaaccctgtctctactaaaaatacaaaaaatcagccaggcatggtggcaggggcctgtaatcccagctactcgggaggctgaggcaggagaattgcttgaaaccagaaggcagaggttgcagtgagcctagactgcaccactgcactccagcctgggcgaaaagagccaaactccatctcaaaaaacaaacaaaaaaacaaaacaaaagaaaacatggcaaagcctttgaaagcttgtctgggagaaggtgcgatgatagttgcataacttcgtgcaagatgctggtccacacaggggctgccccttgctctttctcgctctcttaacctctcatataacaggcttgtgtgttattcacatttattgagcccaagcaggtgcaaggcattgtgatctaatactttggtcagcaagacaacaagatagatcactgccctgcccttaggaagtgtatatgctattagaggaaacagataaaataaacaaggaaaagtatcagacaatgtaagtgctatgagaatgcaaatgaggtgatgtgaattaaaataggatgacttaaagtctgcacgggaaggagcctacccccatgttcctggctagccaaggaaccaccagttgattagcagagaagggcagccagtctagctagagcttttggggaagagggagtggttgttaagagatgagattaaagaagccgagacgggccattcgtgaggggtttgtaatgcagggctgaggagtgtccgaagagaatgggcaggtgagcggtgagacagttgttcttccagaagctttgcagtgaaaggaatcaaagaaatggagccgtgtatcaggtggggaagggtgggggccaagggggtgtccttccccatacagagattgcaggctgagaatgactatatccttgttaacaggaggtgggagcagggcacggtagctcacacctgtaatcttggcactttaggaggctgaggcgggccgatcacctgaagtaaggagttcgagaccagcctggccaacatgcaaagccctgtctctactaaaaatacaaaaattagctgggtgtggtggtactcgcctgtaatcccagctactcgggagactgaggcaggagaatggcttgaacccggaaggtagaggttgcagtgagctgagatcatgccactgtgctccagcctaggtgacagagagagactccatctcaaaaaaaaaaaaaaaatacaggaagggagttgggaatagggtgcacatttaggaagtcttggggatttagtggtgggaaggttggaagtccctctctgattgtcttttcctcaaagaagtgcatggctggtgaggggtggggcaggagtgcttgggttgtggtgaaacattggaagagagaatgtgaagcagccattcttttcctgctccacaggaagccgagctgtctcagacactggcatggtgttgggggagggggttccttctctgcaggcccaggtgacccagggttggaagtgtctcatgctggatccccacttttcctcttgcagcagccagactgccttccgggtcactgccatggaggagccgcagtcagatcctagcgtcgagccccctctgagtcaggaaacattttcagacctatggaaactgtgagtggatccattggaagggcaggcccaccacccccaccccaaccccagccccctagcagagacctgtgggaagcgaaaattccatgggactgactttctgctcttgtctttcagacttcctgaaaacaacgttctggtaaggacaagggttgggctggggacctggagggctggggacctggagggctggggggctggggggctgaggacctggtcctctgactgctcttttcacccatctacagtcccccttgccgtcccaagcaatggatgatttgatgctgtccccggacgatattgaacaatggttcactgaagacccaggtccagatgaagctcccagaatgccagaggctgctccccccgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacggtcagttgccctgaggggctggcttccatgagacttcaatgcctggccgtatccccctgcatttcttttgtttggaactttgggattcctcttcaccctttggcttcctgtcagtgtttttttatagtttacccacttaatgtgtgatctctgactcctgtcccaaagttgaatattccccccttgaatttgggcttttatccatcccatcacaccctcagcatctctcctggggatgcagaacttttctttttcttcatccacgtgtattccttggcttttgaaaataagctcctgaccaggcttggtggctcacacctgcaatcccagcactctcaaagaggccaaggcaggcagatcacctgagcccaggagttcaagaccagcctgggtaacatgatgaaacctcgtctctacaaaaaaatacaaaaaattagccaggcatggtggtgcacacctatagtcccagccacttaggaggctgaggtgggaagatcacttgaggccaggagatggaggctgcagtgagctgtgatcacaccactgtgctccagcctgagtgacagagcaagaccctatctcaaaaaaaaaaaaaaaaaagaaaagctcctgaggtgtagacgccaactctctctagctcgctagtgggttgcaggaggtgcttacgcatgtttgtttctttgctgccgtcttccagttgctttatctgttcacttgtgccctgactttcaactctgtctccttcctcttcctacagtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtgagcagctggggctggagagacgacagggctggttgcccagggtccccaggcctctgattcctcactgattgctcttaggtctggcccctcctcagcatcttatccgagtggaaggaaatttgcgtgtggagtatttggatgacagaaacacttttcgacatagtgtggtggtgccctatgagccgcctgaggtctggtttgcaactggggtctctgggaggaggggttaagggtggttgtcagtggccctccaggtgagcagtaggggggctttctcctgctgcttatttgacctccctataaccccatgagatgtgcaaagtaaatgggtttaactattgcacagttgaaaaaactgaagcttacagaggctaagggcctcccctgcttggctgggcgcagtggctcatgcctgtaatcccagcactttgggaggccaaggcaggcggatcacgaggttgggagatcgagaccatcctggctaacggtgaaaccccgtctctactgaaaaatacaaaaaaaaattagccgggcgtggtgctgggcacctgtagtcccagctactcgggaggctgaggaaggagaatggcgtgaacctgggcggtggagcttgcagtgagctgagatcacgccactgcactccagcctgggcgacagagcgagattccatctcaaaaaaaaaaaaaaaaggcctcccctgcttgccacaggtctccccaaggcgcactggcctcatcttgggcctgtgttatctcctaggttggctctgactgtaccaccatccactacaactacatgtgtaacagttcctgcatgggcggcatgaaccggaggcccatcctcaccatcatcacactggaagactccaggtcaggagccacttgccaccctgcacactggcctgctgtgccccagcctctgcttgcctctgacccctgggcccacctcttaccgatttcttccatactactacccatccacctctcatcacatccccggcggggaatctccttactgctcccactcagttttcttttctctggctttgggacctcttaacctgtggcttctcctccacctacctggagctggagcttaggctccagaaaggacaagggtggttgggagtagatggagcctggttttttaaatgggacaggtaggacctgatttccttactgcctcttgcttctcttttcctatcctgagtagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgaggtaagcaagcaggacaagaagcggtggaggagaccaagggtgcagttatgcctcagattcacttttatcacctttccttgcctctttcctagcactgcccaacaacaccagctcctctccccagccaaagaagaaaccactggatggagaatatttcacccttcaggtactaagtcttgggacctcttatcaagtggaaagtttccagtctaacactcaaaatgccgttttcttcttgactgttttacctgcaattggggcatttgccatcagggggcagtgatgcctcaaagacaatggctcctggttgtagctaactaacttcagaacaccaacttataccataatatatattttaaaggaccagaccagctttcaaaaagaaaattgttaaagagagcatgaaaatggttctatgactttgcctgatacagatgctacttgacttacgatggtgttacttcctgataaactcgtcgtaagttgaaaatattgtaagttgaaaatggatttaatacacctaatctaaggaacatcatagcttagcctagcctgctttttttttttttttttttggagacagagtctcactctgtcacccaggctggagtgcagtggcgggatctcggctcactgcaacctccgccttctgggttcaagcgattctcctgcctcagcccactgagtagctgggattacaggcacctgccccgacgcccagctaattttttgttatttatttatttttttttttagtagagatgaggtttcaccatgttggccaggctagtctcgaactcctgaccttgtgatctgcctgccttggcctcccaaagtgctgggattacaggcgtgagccaccgcacccggcctgcctagcctacttttattttatttttaatggagacagcatcttgctctgttgcccaggctggattacagtgatgtgatcatagctcattataccctcctgggctcaagcaatccccctaactctgcctccccagtagctaggaccacaggcatacaccaccatacccagctaatttttaaaattttttgtagatagatagagtctcactatgttgcccaggctggtctctagcctacttttttgagacaaggtcttgctctgtcacccaggctggatagagtgcagtagtgcagtcacagctcactgcagcctccacctcccaggctccatccatcctcccagctcagcctcccaagttgcttcaactacaggcctgcaccaccatgcctggctaatttttatttatttatttttattttattttattttatttttttgagactcagtctcactctgtcgcccaggctggagtgcagtggcatgatctcggctcactgcaacctctgcctcctgggttcaagtgattctcctgcctcagcctcccgaatagctaggactacaagcgcctgctaccacgcccagctaatttttgtatttttagtagagacagggtttcaccatgttggccaggctggtctcgaacttctgaccatgtgatccgcccgcctcggcctcccaaagtgctgggattacaggtgtgagccaccacgcccggctaatttttatttatttatttaaagacagagtctcactctgtcactcaggctagagtgcagtggcaccatctcagctcactgcagccttgacctccctgggctccggtgatttcaccctcccaagtagctaggactacaggcacatgccacgacacccagctaattttttattttctgtgaagtcaaggtcttgctacgttgcccatgctggtatcaaacccctgggctcaatcaatccttccacctcagcctccccaagtattggggttacaggcatgagctaccacactcagccctagcctacttgaaacgtgttcagagcatttaagttaccctacagttgggcaaagtcatctaacacaaagccctttttatagtaataaaatgttgtatatctcatgtgatttattgaatattgttactgaaagtgagaaacagcatggttgcatgaaaggaggcacagtcgagccaggcacagcctgggcgcagagcgagactcaaaaaaagaaaaggccaggcgcactggctcacgcctgtaatcccagcatttcgggaggctgaggcgggtggatcacctgaggtcaggagttcaagaccagcctagccaacatggtgaaaccccgtctctactaaaatacaaaaattaaccgggcgtgatggcaggtgcctgtaatcccagctacttgggaggctgaggcaggagaatcgcttgaaccaggaggcggaggttgcagggagccaagatggcgccactgcactccagcctgggcgatagagtgagactccgtctcagaaaaaaaagaaaagaaacgaggcacagtcgcatgcacatgtagtcccagttacttgagaggctaaggcaggaggatctcttgagcccaagagtttgagtccagcctgaacaacatagcaagacatcatctctaaaatttaaaaaagggccgggcacagtggctcacacctgtaatcccagcactttgggaggtggaggtgggtagatcacctgacgtcaggagttggaaaccagcctggctaacatggtgaagccccatctctactaaaaacacaaaaattagccaggtgtggtagcacacgcctgtagtcccagctactcgggaggctgaggcacaagaatcacttgaaccccagaggcggagattgcaatcagccaagattgcaccattgcactcccgcctgggcaacagagtgagaccccatctcaaaataaataaataaatatttttaaaagtcagctgtataggtacttgaagtgcagtttctactaaatgcatgttgcttttgtaccgtcataaagtcaaacaattgtaacttgaaccatcttttaactcaggtactgtgtatatacttacttctccccctcctctgttgctgcagatccgtgggcgtgagcgcttcgagatgttccgagagctgaatgaggccttggaactcaaggatgcccaggctgggaaggagccaggggggagcagggctcactccaggtgagtgacctcagccccttcctggccctactcccctgccttcctaggttggaaagccataggattccattctcatcctgccttcatggtcaaaggcagctgaccccatctcattgggtcccagccctgcacagacatttttttagtcttcctccggttgaatcctataaccacattcttgcctcagtgtatccacagaacatccaaacccagggacgagtgtggatacttctttgccattctccgcaactcccagcccagagctggagggtctcaaggaggggcctaataattgtgtaatactgaatacagccagagtttcaggtcatatactcagccctgccatgcaccggcaggtcctaggtgacccccgtcaaactcagtttccttatatataaaatggggtaagggggccgggcgcagtggctcacgaatcccacactctgggaggccaaggcgagtggatcacctgaggtcgggagtttgagcccagcctgaccaacatggagaaaccccatctctactaaaaatacaaaagtagccgggcgtggtgatgcatgcctgtaatcccagctacctactcgggaggctgaggcaggagaatcgcttgaacccgggaggcagaggttgcggtgagctgagatctcaccattacactccagcctgggcaacaagagtgaaactccgtctcaaaaaagataaataaagtaaaatggggtaagggaagattacgagactaatacacactaatactctgaggtgctcagtaaacatatttgcatggggtgtggccaccatcttgatttgaattcccgttgtcccagccttaggcccttcaaagcattggtcagggaaaaggggcacagaccctctcactcatgtgatgtcatctctcctccctgcttctgtctcctacagccacctgaagtccaaaaagggtcagtctacctcccgccataaaaaactcatgttcaagacagaagggcctgactcagactgacattctccacttcttgttccccactgacagcctcccacccccatctctccctcccctgccattttgggttttgggtctttgaacccttgcttgcaataggtgtgcgtcagaagcacccaggacttccatttgctttgtcccggggctccactgaacaagttggcctgcactggtgttttgttgtggggaggaggatggggagtaggacataccagcttagattttaaggtttttactgtgagggatgtttgggagatgtaagaaatgttcttgcagttaagggttagtttacaatcagccacattctaggtaggggcccacttcaccgtactaaccagggaagctgtccctcactgttgaattttctctaacttcaaggcccatatctgtgaaatgctggcatttgcacctacctcacagagtgcattgtgagggttaatgaaataatgtacatctggccttgaaaccaccttttattacatggggtctagaacttgacccccttgagggtgcttgttccctctccctgttggtcggtgggttggtagtttctacagttgggcagctggttaggtagagggagttgtcaagtctctgctggcccagccaaaccctgtctgacaacctcttggtgaaccttagtacctaaaaggaaatctcaccccatcccacaccctggaggatttcatctcttgtatatgatgatctggatccaccaagacttgttttatgctcagggtcaatttcttttttcttttttttttttttttttctttttctttgagactgggtctcgctttgttgcccaggctggagtggagtggcgtgatcttggcttactgcagcctttgcctccccggctcgagcagtcctgcctcagcctccggagtagctgggaccacaggttcatgccaccatggccagccaacttttgcatgttttgtagagatggggtctcacagtgttgcccaggctggtctcaaactcctgggctcaggcgatccacctgtctcagcctcccagagtgctgggattacaattgtgagccaccacgtccagctggaagggtcaacatcttttacattctgcaagcacatctgcattttcaccccacccttcccctccttctccctttttatatcccatttttatatcgatctcttattttacaataaaactttgctgccacctgtgtgtctgaggggtg

SEQ ID NO: 6 - protein, 393 aa, Homo sapiens [sequence omitted]

SEQ ID NO: 7 - DNA, 31344 bp, Homo sapiens
gaaagaaacaacaactggaaaagaagcactgcataagaccaggatgtctctgaaatggatgtcagtctttctgctgatgcagctcagttgttactttagctctgggagttgtggaaaggtgctggtgtggcccacagaatacagccattggataaatatgaagacaatcctggaagagcttgttcagaggggtcatgaggtgattgtgttgacatcttcggcttctattcttgtcaatgccagtaaatcatctgctattaaattagaagtttatcctacatctttaactaaaaatgatttggaagatttttttatgaaaatgttcgatagatggacatatagtatttcaaaaaatacattttggtcatatttttcacaactacaagaattgtgttgggaatattctgactataatataaagctctgtgaagatgcagttttgaacaagaaacttatgagaaaactacaagagtcaaaatttgatgtccttctggcagatgccgttaatccctgtggtgagctgctggctgagctacttaacataccctttctgtacagtctccgcttctctgttggctacacagttgagaagaatggtggaggatttctgttccctccttcctatgtacctgttgttatgtcagaattaagtgatcaaatgattttcatggagaggataaaaaatatgatatatatgctttattttgacttttggtttcaagcatatgatctgaagaagtgggaccagttttatagtgaagttctaggtaagtcgtgtgtccaattggtgtttattaagttctaattttcctgtgcctttgaaggtgggcttatataaatataatgtcagaagatagtgtttttatgggaaattatgaattgcaaatgtaagatgatctatgagtctcaaaaatatagaatgttgaccttatagaatcagttagaaccctggtgccatcactgctataggacaccaagagagtcataaaccttcaatgtaaaacacttatgatttctttaagccatcacatatcattttgctatacattttttcatctttaaaaaagtcaatagatacttcaagaaacatcttcatgaaggcagacatacaaattttatatttacacatatttctaaaaatattatcaatgcaggattgaggaacttgtacctgagtacctcagtttcctcatttagaaattaaattttgtttttcatataagaaggattccttcacagttgagaaatatagtggctctactccagaaacagaagcctaaaacttgagatttctaatatttatacattccttcaataacaacttcacaattatttccttcaaaaactgaaatcttgttgaaagtgaacatctaagttttaatctatattttattaaactgcatctctccatcaaagaaaataggggccaaaataaggaagagcacatatctctatgtcaataaattctgaaaaatttttaattctcatttgtaaatatatttattttaaaaatctaattatattaagatcttaagatgaaccaagacagtagtaggtgtaaagatttcagtgttgagctcaaaaaactcatggtttactttgagaaccaaggatcaagggactagcttaataaactgtagacactagagtacttcctggaaagctgttttcatgggtaaggtaagatgaattaattgtggaactgaaagagttgtttaaaggtatatttgttactattgcagcttcagagggaagacaaatgtgtatttaagttcatagtggctacattagtccattctcacactgctgtaaagaaatacctgagactgggcaatttataaagaaaagaagtttaatagactcagttctacatgactgggatgcctcaagaaacttagaatcatggtgaaaggctaaggggaagcaagcttggatcttctcacttagtggcaggagagagaagtgcaagcaggggaaataccagacacttataaaactatcagatctcatgagagctcactcaatatcatgagaacagcatggaggaatccacaccatgatccaatcacctgccactgggtccctccctcgacacatggggattatggggattataattcaagatgagaggagatttgggtggggacagtcaaaccatattagtgacttattttaataattatttatgattgtgaatatactgatgttacattaaagatgtgatttcttcttacagctctctgaacactttgccttccttatatatacatatgagcaacatatgcaatacataaaaattaaatgatgactatataaatgtatttaatataatgtatttacattatattaaattattttattaaataatttaaatattaaattaaaactattaaatattaaatattaaattaaatattaaatattgtttaattttattaactttattaatgttttattaataaaataaaattaataaaattttattaatattaaatattaaactttattaaatattaaaaattaaaaattaaatactattaaattaaaatatattaacatatattaaatacattaatttaatataatgtatttaatattatttaatatacattaatttaatataatgtatttaatataatgtattatattatataaatgtatttatatatattatcaatgtgcagacattttatatatttttaggtatgttattccaagtcctttcaggaaaatacctgcatattcaatattaaaataacaattctcgtgttagctaccttttgttttgttttgtttttttccatcaggaagacccactacattatttgagacaatggggaaagctgaaatgtggctcattcgaacctattgggattttgaatttcctcgcccattcttaccaaatgttgattttgttggaggacttcactgtaaaccagccaaacccttgcctaaggtgaacatattcttgtttcatttgtttgcttgacattttcagaaggaatggctggatatgtttctttcagagtgtttaactcagagtgaggggaatatgggaggtcaaaaaaaaggacttgccattagaaaatcatatatttctatactatcacaagtatgtgaatgttattatcattaaagaccaaagaggtttactagggagattttgaaaacagggttggttaaagtaaggccttcattgtgcaacccacaagatagtatggttcatttcttcaaaaaatatttgtagagtgattaatgcaaaccacaggtaagtgctggattttcagagaataaaggtagcacagtttctgcgccctcatgcctaacattgtactttgaaagatagaataaaaacaagtgaaaaagaaaagtctaaaaagtgttataaggaaagaccacaatgataaagaaatatgcagaagagatcccaaactcattgacaattaaagtgagtactcaataatgtgcagagataggtgaaacgatgaggggatgagaaagacccaaaaagaaaaccagaggccaggcaaggtggctcacacctataatccctgaattttgggaggcagagaagggacgattgcttgggctcaggagtttgagaccagcctgggcaacatggtaaaacctcatctctaacaaaaataaaaataatagccaggcatgctggtgtgtgcctgtagtctcatagtcccagctacttaagaggctgaggcaggaggcttgcttgagtctgggggcagaggctgcagtgagctgagaccacaccactgaactctagcctgtgcgacggagtgagactctgtctcaaaaacaaacaaataaacaaacaaaaaagtaaagtcttgcattaaggcagaaaagtagaaaagcagtaagggcagttcccaggactccaaatttagtttacaggaaaaggttgagtaaaagtcaatgatgggccgggcgtggtggcttacacctgtaatcccagcactttgggaggccgaggcaggcggatcgcctgaggtcaggagtttgagaccagactggccagcatggtgaaaccccgtctctactaaaaatacaaaaattagtcaggcatgttggcaggcacctctaatagcagctactcgggaagctgaggcaggagaatcgcttgaacgcgggaggcagaggttgcagagagccgagatcatgctattgcacaacagcctgggcaacagagtgagactctgtctcaaaaaaaaaaaatatatatatatatatatatatgaaatttgatatacccattaaggacaatggagagctactgaaaagagttaagaaatgaagagatatgatcagatttcctttcaaaaaaaatcccaatgttctcaatgtgtaatattacagaaaggcaactttgtacaatgtaaagaatatttaggaacttttacatgagtttaggcaatcatgactacttttacaataataatcacaacttcatattgtgttgtgtggaaaaaagtagttaccacagataaaacacttaagttgtctctgacacatagccagtgatccaaaaatattgttgatattatgattattattttagtcattatttctaatattttaaattagctaacatgtgccagccatttaagatatcagtcttcatttaataatgattgattttcctagcaaaatcctagagaacagatactctgtgtgcttgtttaaatataaatgtttaagtttgggcagtcagttgaacacgctggaaagctaaagttgtattgcaaaggtggaggcacttcattaatattttagcttaaaaggtaattctgtgtactcttatcttattctcaaaaaaaaatcctcactcacatcaaaagaaagtgatagtgccattagcagggggaagagaataggagaataaggaggacaaggaataaatgactagtagtacaatagtgattattactaatactaccatgatctcagctcactgcaacctccaccctctaggttcaagtgattctgattctcctgcctcagactcccaagtagctgggattacaggcacccaccaacacatccggataatttttgtatttttagtagagacagagtttcgccatgttggccaggctggtctcgaactcctgacctcaggtgatccacttgcctcaaccttccaaagtgctgggattacaagtgtgagcctccgtgcccggcactggctgttgttgttaacctgcaaaaggtagacttgaatctaagtaaataattgtgaaaccgattctctaattcttttgtacacaaaataattgttgccacaaatttgcttgcttttccattatgtattagattctcagataatgtttgtatatttcaaaagaataagactcttgccaaaaagtatcaagtgtttgaaaactgcatatagttattgcctttgtaatatattcacatgaaactgtacagagaataaatcatgatggtaagattcaatattagtagcaaatattcgttaaaaaactctggaaataattcaaatatcttacattaagaaatggttaaaaacttatacaatgcgcataccaagccattacaatccattttttcaaaacaatgtttaattacactgtcagctgtagtacaggtatagttggaaactaaggaacagaatgatgagtagaacaagatcacaatttttttgtaggataaaaaggcatatacaatgagaaaaaattttctaaataaacatcacatatgtacattgagttatataaatagaattaaaaccataatgaaatggtacacattttaaattgagatagagatatgggaaaaatgatttttttcctaaattttcatacttttacaagactacttacattgatcttctattaccattgctttcataaagacgtcacagaatgagatcagtgctgatctgtctataaacagacgattttttacttcaatacttggtggatgaagaagacttttgtcagtgaatgagacaaaacattaaaccgaagttacattaaatgtggctacaggcaactgcaattacactgagttcctggatgcctcatgaattaactctaatagccatacactgaatctccaattaaaactgatatcagaccgggcacagtggctcaagcctgtaatcccaccactttgggaggccgaggcaggtggatcatgaggtcaggaggtcaagaccagtctggccaagatggtgaaacctgtctctactaaaaacagaaaaattagccaggcgtggtggcaggtgcctgtaatcccagctactcaggaggctgaggcaggagaattgcttgaacctgggaggcagaggtcgcaatgagccaagattgcatcattgcactccagcctaggtgacagagcaagactccatctcaaaaataaataaataaataaaataaaataaaaataaaagatgtcataaagttcaattatatttttgaaaaataccaatatatggtctgaattgacatattttagaatttccttttttattcgtaaacagttttataaagaaatttcccccaatgattaggaccagaaagtatatttgttatgatagaaggggttggtcattattttactgagaaaaaaagaatttagaacaaaagggacaatagcaacaaaaagatgagtatacaattttgatatgatatccgggaaacttatgagtctacatttcttttcctctttctctctttctttttctttttttttttttttggtgtgagaatacattgactgatgtggcattagagaatggatttaagtttaaaaacaggaacacatcaggaaacacaagtcagaataattctcattcatttcgaagcaaacacatatttaccaggagcttcatatagtgtgagggagctactctagggggtgagcagatctccactggagaaaggcttggtgacctctcccactgtggttcaagtgccccctgtgagacacagcaaagtgaggatgagggtccctcatggtaagttataaatagcacatcaatttcacagtgtgatttcaggacaaaaggtatcatagtcatacctaaacaatgatctgagaaataagatcacttaattatgtaactaaatagtatataatactgtattataaatggaattctcagaactatttcccggaaattccaagccccaacaccagactgatgaacgtcaatgattcttatacttcagcttttaaggtgttttttgggggtgggggtgtcgggggaagtgaggggtagtggggtaagactaggaaactctaagtgaaatataaagggttagagagctaaagaagctaaataaatgtagatataaaatcattctaattgtaactaacttttcctcattgctcatagtcatgtaatggctccaatgactcctaactaaaagaactgaacagaaaaaaataaaataaaatgaccccaaataaacacaagagatttagtagaactacaaaaaaaaatagaattaaagccaagtaaagctaccttccaataacctatgttagtaattataatattataaataaagtgtctgggtgtaatattttctagcattatctcctcctactgcagtgtcatagctttgtttcatttcttccttacacacacatacatacatgcatatgcacacatatttacacaaatttgcatgaaagatcccacatatattatattacacatctcctttcaaagaaactgaatgattaggtcagtttaaaaaaattactccaatagctcctgactttctcatcttagatgtttgtaacaatcctgtcaacagtgttttctgtgctgttgctcttttctgatagaacaaattctttcttcacaggaaatggaagagtttgtgcagagctctggagaaaatggtattgtggtgttttctctggggtcgatgatcagtaacatgtcagaagaaagtgccaacatgattgcatcagcccttgcccagatcccacaaaaggttagataaagtgccttaactgtggatggctactaaatgaatctgttaaactcttcaagagtccattacagaaatgttctgcctgaaaatttaactgctatgatagttctaattatctcagacatctgttcaaagcaaaaacatatatggaagatcttaaaatcataaagagaggagttttggttgataataacgttggcattaatattgtgatcagaaggaaatatatttaagaggtgctagtgaagtttggtattatcatggtatcgtagcatgtacatagaaatcactaaattctgccctgtcatttgctccttttggtttacaggattttagaaggtactgtatacactgtagatattatcaaaaagcggtaaaattttaataagttactgtactatcacaataacaataagcaggtattgaaaaaactttgaaatgcatcatgcagcttcgtcttaccaagcaatctggctgtttttacttcccatgcattggaataggtctatttagcgttctgttcagggtgccattcagagaaagaatgtcctagtctgactagccactgctctggaggtacccacctagccaagtagatttagagaaaaagaaaaacttgtctgctctgctggcttgggtgccgctaatgtcttaagcagaaaaatgtaatggaaaggatggagatagaatcctgcttttagggtagactatgtgcattattaaatgtggcccaacaaagactatgctctaagaatcagattagtgccttctccacattgaggaaggagcaggaactataaataaacatggtaggaacttattttctaatctatagaatatacctacaatctttgtgtattttcaatttacgttttactttcttatctgcatagccctctctgaatgatctatgcaagtttttgctgaaaacacagagttactttaacactcccatatcaaatacaaggacaacacttgtaaattctactccagtttataaagattgcttgggaaatctaaaatcagtaccttagtttggtactagacatggtaatgactggctatagctgaccatatatcaaggctgtcaatgctaggtagtgcatcatctaagtgtgaaagatcagttaagcaaaaagtagcaaaatacaattttctacattctcagcatctgtattgttaatcatgaattgtcaatgggctcctttttcctacattgttagcatttacaaaagtagcaatactgataatagtctgaatttactacatatgtagcattatttctcaccatatgtgcagtttaagtatttagtcttttgtaatcaatgatgtcaaattcataaaataacttatgactagtagacaagtggatataagtaaactcttcccatatcactcaaaaattaataaatttatattaaatttggaaggttacagctagtataaagtatataaaataaatatataggttattactaatttgctacttttttattactagaattaagactgcctaacaattcttatatatttgaatatttcattgataatcttgtgaaccccaaatatctgatacaggtctcagtacatttagaaagtttattttgccaacttgaagcagggaagaggcattcctttgagcttctgattaggctctccaaaggaggcaatcagatatgcatttatctcagtgagcagaggggtgtctttgaatagaatgggaggcagtgatttgcttagtgactttggggcccccaggtttattttcattttacattttaaaaaaatcttttggagaaagcattttagaagaaaatgagtctctggtctcaggttttcatctgatctctaatggcaaggaaggtttattcctaagcaggtcctgagttattaggaaagttcatttttagcaggttctgaagtctcatgtactgtgaagacaaaataaagggaggaagtgagaaaaacaaaaacaaaagaacaatcctttaaaattaatataggccacattactctgaagtccataaatcagtaggcaggtatgaaagtggattatgtatgtaaattggttgctgttattttcttctggagtttaagttgtctagcttaatttgtagggctttatgaaagcatagcttagtttacagtgactccaaattaggaaaaattgtggaaggggaggaaaaaaaacctgaagacactattttgaagacgtgtagccatgaaaaattagaattcagtccaaactgtagacagtaataaaaattgaaaaacattaggcaagactagaatctaacaacaggtatactgttatttttgaaacataatttttctgtcttcagtttcccaattttactaaagacaaaccatggtacaactgatttgctttattatacttggcctgattattgtatacagaggaggaagaataattttttatttacataggcttttaaagtggctttgatggaactttgtcttatagaaagaatctcagataggacttttttaaagctgaggcaagccatagatttgtaccatcaaatacctatgagttgggtgaattcctctcctcttggggttccaaaataaacttggggctcctgggcctgtcagaaagttacattctttgcttaacacagttcaggaaccctgtacagggaccttatagatgaaggtatgaggccagttttcccaaggggttttattggctccataagttaagtttgattccctaaaggaaagcccaccattccagtggaagccttgataaaataaccagtttctccaattgtgttttgttaaaaatgaaaatagattcttattgcacttatgcaaataactgtattgacaaaagtaaaaaatactcacaaatagtttccaaattctgaaaaatcaggtagagggaaacaaatatgttctaaatttttttcataggagtatactaaattgttaaaagctgttaatagctcaaaaaaagaaagattccttcactctaaaaaaaaaaacaaaacaaaacaaaggatcagcaatgttttaagcaaagtcaaaaagattactttagttttctattatttcagtccatgtagttaattcctgttctgcttgatattcatgaacattttagttctccatgagtcctgaaagtttttcttccattctaatgtcacaatctccaaaattatcaaaaactttttttcaagagcacctgttagaatttcatggctgagtataaaatcgccttctaaagaggaccaaagtcagacaacaattgtctgtggatgacaaaaagttttagggcagcctcagtcaaagacacaatggacaaggaaatttgttacctctgtggcacacaataacttaacataacaaatataattattactgataatgtacacactaagtcatatcagaattataggagtttcccataattttggaacacatgccaataacatatttatataaatacagctcaaaaagaccaaacaccatttcatgtttgacaatgttccctgtataatttttataccaaataagccaaattatgtcatttttggactttagggaaactaataaattaaaggattacttaggtcagaaaaaaacataattttggaaaatttgtcaaatatcaaaggattaaaaagcttgatatcacaggtaaaataaagcattcatttgactaaagtagtaactcaaggatttcaaaaaaaggcaaaaatcttcattctttgagagaggagactcaatttgccaaacaataagccctaataaaacagcatgaagccaaataagtttgtttttcaaaattgtatgaacaatctataaaattctaatcttgaccataagatataactttcctcagcctttcataacgtttataatctttattaagaagtcagtttatgcttcaagaaaaccttgttaatctgacacaggggcccatatgttggtcttgcattagtgtgtctttgacattaatgattaatttatgcataaacggaacttattttatctcttagtatcagttctttcaatctcacacacccacctcttctgtgatagtccctggaccttgaggagttgaatagcttttagaaaacactgttagcattatgccacaacaaacagaacttgaggaaaaaacttacatgagttgaaaatgagttgaaggagagtgttactatttcacaccctttaaaaggggagagaaaaccaaaaacagtgagatgcaagaaaagttaaactttgggttaaaaaaaaaaaactcataatattttaagagtaaatcaatcccttaagaaaatgttattgttctaaccaattatttagtgcataagtgctttttttacatcaagcccaatctgtagaaacaccattataatttccctttaattatagacaacttgatcatataaaagtttttttaatcttcttattgtgacttacacagatcattcatgatatgtttggactttctggtttgtcctgaacatccttctttcttacacaactgatcattttattctaggactaaatttaccacacaagattctttctcatataaaattatttctcttcaagctttttacaaaaaaaaaaaaaaaaaactttttatttttataactttctttacatctcccttacttcctggttccttttaccttgttttatacataacctttaaataagctttgaattagacaacacttcttcacctttttaaaaaagatgcacttttttttggaaagaatgttttcctacaaatttttttttaaaaatacccaaacaatgaaatatctattatttaatttaatataaccttagattctaaattatgagtttgtctataagtatttatcccattacatttacctaattatttagtcatttacctagaatatttgtgaaaagtgtggtagtcatcatttaaagttatggaattgccattgcaaaattataactgagacagtgaaaaagatctgacctaactccatcttgcttttaacctccaagctctccgtgtttatttctgggtgtaggccaaactaacttaaggaggaacttagtttatagtttagctttgaaataaaaatgataacagtcttttcccaaaacaaagcttaatgcctgtggactagattgcttaaggccacaagatgagaagttatgataatcttacttaattcaaggtgtagctatttgcattaaaccaatattaatgtcttatttattaaaaattacacaagtgaagatcattctgttttgggcttggtttacagttttgtaacccctatgtcaaattttgacaccttaaagtatttggcaagaataagtatgaaatttcttgattaataaatgcaaacaaaaatgtatgctggcaagtcttaatgcatttctaatattatcatactttatcaataattgttgttatcttatttataaaagattttatttaagttacataaacttgaaaaagcatttaattagtctttttttctggcaaagtatttaattcaagcacttttattttcttaagccaattaattagagctcttttatatattttcagtaataaaacattgtgtacaaaacacataaatacatagatgtagtaagcatgccaacagaagtacatcttacaggtttataaaaactcctctttgccatattagactttcaaattcttgataacctgtttcaataggtaatctgtatgaatacttgatcaccataggcagttgtgagctaaataatcttacatttgcatattaaaagaaaccattcaggtgaaaatcaaatagcaaaatttacatcataatgtacacagagaaaaaaattggtgtgctagaaggaaaataaaatggatttaattgccaattaaacataaaattatagaaattataatggccttttaaatatatgcaaacacttacacacacacaaaagattatgtggtttttacttcagaaatttagctatgagataaaaaaaaatcaccagcttgcagacaaaaacctgttgaatcaaaacagtggtttttaacttaatagaaaaataaaagcaaatttaaagcaagaataaaagaaaatttttaaaaaagagagaacttaggaactctacagtttgtaggacaaccttagggctcttaatgtatatgtgcacaaagaccgtattaccttcattttacataaactctgccaagtagaggtgtcataaatccaaacaggcgatatcaggggatcattcttcttcttttctcatcattcttagattatttgtttcccactttttttcctaaaaggaggaactgagctgtggcctagggtttatgtgtggtgaatcaatgtgtgctgcttgtgggcagtactccacagtgtgtcaccactgagttgtttccaccctcttacgtgtctcagtttctccctccagaggtgtatgacctctgagagggctcaaaatgctgggtgatctgccctcatatgtgtttcctggactagcctttttttaaaagttaatttttcttggggatttccctgcaggaccactgcatgtcatagggggtcaaacccaagacactcccacaaggcccccagttgcttaggggtgccttttgcctgggaggagcagatgccctttcactttggagctgagaaaactcagtctttcattttcctatgtaaaccacagttcagtttctcacacaaatccacacagacaagccaaatgagattaatttggggaataaaaacaatagaggagaccctttacaacacatctccaaactagaattaggattcttaaacaacaacttcctaggaggaaaaaaataacaacagtcaatactacttccagtaaactgtactcagccaccctctactttgtaactcgtctgccattacacacgccaaggtaaaatcctctcacagtacaaggtaatctgtggtaatctcgaagccaaagagatcaggtcattcaacataggaaaacagagctttggacctaagaaaaaatctgcccacaactcttgaaactccacaaagaaaacagagcacccccaaaggggtgagtggtgcctttgttctgaattctttaaaagagttcaaatcattagaagccttctctagatttttggtactacaaatggcaaaagacaaaaggaggtatagggtggaggaaaagtaaacaaaagaacatttgtttttttaaagacaggaagcaaacacagaaaccaagagcatggtttttaggttttgttttgttttctcttttgcatctgtgaagaattttcaccaaattagacaggctttgttacccacaatttggaattctcactcagatatgatcaagtcaggtaaagtttgtcaaatctgatgggagaaatactggaatgtacaaaaaaaatactcccaaaaatgtgatcattaagcactgtaatggtaaggagaaattaagtccagctagttgttaaacattaaccaagacaaaaccccaattcagctatttacctacggatgggtttcaggctaaaactgccctctgtcgtcctagaagtaggaaagaaacctcaaatttatcctccctcctgggagtgagctcaaactccatagagttacctgccttccattgtcatggaaacaggaaatattgcctttcttgtaggaagcaagtaaaactccaaaaaaaaataaaaaacaaaaaaaaaggagttgtacagcaaaataaacttttggtcttgactgaattttggggtatcagggattctctggaggggttgctcccagacctcagccaattgtcttatttgtttgagccataaagttagctcatgctggtaccaagcactgacaggacatttatcaaaggtcagggaacctccactcagagtctcttcatggttaccaaaatgtgaaccctgaatatctgaggcaggtctcagttaatttagaaagtttattttgccaaggttgaaaatgtgcacccatgacacagcctcagggggtcctggtgacatgtgtcccaaggtggtggggggacagtttggttttatacattttagggatacatgagacatcaatcaacttatataagatggacatttgttcagtccagaaatgggggacaactagaaacaaaggcagagcaacttgaagcatggaaggggcttccaggtcataggtagataggagacaaatggctgaattcctttgagtttctgattagcctctccaaatgaggcaattagatatgcatttatctcagtgagccaagggctgactttgaatagaatgggaagtaggtttgccctgagcagcttaacttttccctttagcatagattttggggcaccaagatttattttcctctcacactctcatgtgtctagctattaagttagaaatgtcatttttttatataccaaattgattataaaagtaaagatattaaatgtgggtattcagcttacctcagacttttagtagttctcattacttgacatcacttcttcttatttcttcatcttttatatggattaactgattattaatctcttcagaattctaacaagttatatttttagagttctattcattgaacaagatattttccttgccctaacaggttctatggagatttgatggcaagaagccaaatactttaggttccaatactcgactgtataagtggttaccccagaatgaccttcttggtaagactctggagaacaaatactgaatatattagtaacagccaattagaagtgataatatttcaacataaaacaaacatatttaacatttattattggaaaactaaaaaaaaatcaaatttaactactttatatttattttctagtcttagtataagaagaatacactatggtagttggcattttgttacatacagtcacattctttatggtcagaataaaaatctctttattcaggtgtaattacctctcacaggttttaaataacatcctggattttctgtctcttatttctgcaactttacatctgttccttcccctactgcagggttatttcaacaggtactgaaaaataacagacattcttctattaccagtgactctattttctgtgggaataaatcaccaatctttatcataaagtgataacacatttcatgatgatgtgtaacctgtccttcctcagcaccacctccaccctactccttgctgcctttaaaaaaaaatttaagtattttaaatattttaactatttaaatattttttaaatatataaatgtgactgcataatttatactacttaagagactatgttcttgtataccaaatcttattcttttcttttttgcacattttaattttttaattaagaatatgttttttcattttgttcacctggcaaacttttaaatttatgtttccactgtgaagacacacagaccaccaccttttcatgtagccttcacttacctttccagcagaagtaggtgcttcttcctctgaaatttgaaaacaatttcaatgcagttttgtgggtataatgttacctagggaacagttttgctttaagttccttatattgtgcatttcttattcaattcctgtgccttataattaataactttgttaaaatgcatccacttttaggtcatcccaaaaccaaagcttttataactcatggtggaaccaatggcatctatgaggcaatctaccatgggatccctatggtgggcattcccttgtttgcggatcaacatgataacattgctcacatgaaagccaagggagcagccctcagtgtggacatcaggaccatgtcaagtagagatttgctcaatgcattgaagtcagtcattaatgaccctatgtgagtattacaattttgtgaccaggtggtacttacaaattattttgtcaacagtgaatatgagtttgaaccctttgttaagagactaattttgaagggattgaagtgatttaaccaatgtaaaatctgctcttattttccaccagacaattatttcaaagttacatttcaaccccacagatttaatgggtcaccaatgactgcaatgaattataaaatcaaaaaaattaaagatatgtacataattattttaaatatttttaatgatagaatacacaatagaaattatgtaactagagaaatatgataaaatatttcaattcaatacctaaaatttctgaaagtatgaatctattctttctcaaaaatttatttttattattatcattattttaatcaatttgaaaatgagttacaatttgcatagcataacattcactaaaatttaaaatctaaatatatttagtatgtttacatagttacgaagccacaaccctaaataacatttttgtgagctcaaattaaactcaaaatgcatgaagtcactcccaatacacctccagtcctagaaaaataccgacttactttgtttctcaatagattattctcttcacatattttatataaatgaaatcatacaatttatagtgttttgtaaacgacttctttcacttagcataattatttttagttttatttatgttgttggatgtatcagaactcattcatttttatttctatataatattctttgatatgtttataccaaaattacccatttatcagggcatggacattttaggtctttccaattttggcacttacgaatattgttatacatattttctgtgtaaaaatatgtttttacttcttttgaggagaggctaggagtggacttgctgaggcatatgggaaattgttgtttatgaagaatggctaaactgtttttcaaagtatttgcatcatttttcattaccaccagctggataggagggctctaatttcttcacactcttcccaacacttgttatcacctgtctttctattacagcctttctagtgtgtgtaaagtggtctctcattatggttttcatttgcatttccttatggctgaagatgttaaccctcttttcatgtgcttactgatcattcatgcatcttccttagataaaatttctattttaatcttgtgaacattttgtcatttttctatttaccttattatggattcataagaattattaatatgttatagatactatcccttatcaaatatacgattttcatatattttcttccactatgggcagatagatttttttttttttttactttttggaggatgccttttgaaacatgaaggttttaatattgataaaataaatatgtaatagttttccataatccaagggaaataatatggaaaatgctgtgactacaccagctaccatcaattcagagtctattaatcctaccaatattattatgatgtaaggtggtaccagctaagagaatacattaaaaacactttaccaaaaaattagtaagacaagaaatgatttaaaatacttcaaataaagtaaaaaagcaaagaaacagaaaacaggttagaaaacaaaaatatatagaataaaatggtggatttaaatgtgaactagggcaatcaggcaagaaaaagaaataaagggtattctataaatagaaagagaagaagttaaattgtctctatttgcagatgacataattctatatttagaaaaccccttcatctcagttctaaaattccttaagctgataagcaacttcagcaaagcctccggatagaaaaatcaatatgcaaaaattacaagtattcctatacaccaacaatacacaagcagagccaaatcacgaataaattcccatttgcaattgcaacaaagagaataaaatacctaggaatatgcctaacaaggtacgtgaaggacctcttcaaggagaaatagaaaaccctgctgaaggaattaagagaggacacaaacaaatggaaaaacattccatcctcatggataaaaaaatcagaattgtaaaaatggccatgctgcccaaagtaattttttttattatactttaagttttagggtatctgtgcacaaggtgcagctttgctacatatgtatacatgtgccatgttggtgtgctgcacccattaactcgtcatttaacattaagtgtatctcctaatgctatccctccccactcccgctacccccaagcaggccccggtgtgtgatattccccttcctgtgtcctcgtgttctcattgttcaattcccacctatgagtgagaacatgcggtgtttggttttttgtccttgcgatagtttgctgggaatgatggtttccagcttcatccatgtccctgcaaaggacatgaactcatcattttttatggctgcatagtattctatagtgtatgtgtgccacattttcttaatccagtctatcattgttggacatttgggttggttccaagtctttgctattgtgaatagtgcctccataaacatacttgtgcacatgtctttatagaagcatgatttataaccctttgggtatatacccagtaatgggaatgctagataaatgatatttctagttctagatccctgaggaatcgccacactgacttccacaatggttgaactagtttacagtcccaccaacagtgtaaaagtgttcctatttctccatatcctctccagcacctgtggtttcctgactttttaatgattgccattctaactggggtgagatggtatctcattgtggttttgatttgcatttctctgatggccagcgatgatgagcattttttcatatgtcttttggctgcctaaatgtcttcttttgagaagtgtctgttcatttccttcacccacaagctgatgaggttgtttgtttttttcttgtaaatttgtttgagttcattgtagattctggatattagccctttgtcagatgagtagattgcaaaaatgttctcccattctgtaggttgcctgttcaatctgatggtagtttcctttgctgtgcagaagctctttagtttaatgagatcccatttgtcaattttggcttttgttgccattgcttttggtgttttagtcatgaagtccttgcccatgcctatgtcctgaatggtattgcctaggttttcttctagagtttttatggttttaggtctaacatgtaagtctttaatccatcttcaattaatttttgtataaggtgtaaggaaggtatccagtttcagctttctacatatggctagccagttttcctagcaccatttattaaatagggaatcctttccccatttcttgtttttgtaaggtttgtcaaagatcagatagttgtagatgtgtggcattatttctgagggctctgttctgttccattggtctatatctctgttttggtaccagtaccatgctgttttggttactgtagccttgtagtatagtttgaagtcaggtagtgtgatgcctccagctttgttcttttggcttaggattgacttggtgatgcgggctcttttttggttccatatgaattcaagtagttttttccaattctgtgaagaaagtcattggtagcttgatggggatggcattggatctataaattaccttgggcagtatggccgttttcacgatattgattcttcctacccatgagcatggaacgttcttccattggtttgtttcctcttttatttccttgagcagtggtttgtagttctccttgaagaggtccttcacatcccttgtaagttggattcctaggtattttattctctttgaagcaattgtgaatgggagttcactcatgatttggctctctgtttgtctgttatgggtgtataagaatgcttgtgatttttgcacattgattttttatcctgagactttgctgaagttgcttatcagcttaaggagatttggggctcagacgatcaggttttctagatatacaatcatgtcttctgcaaacagggacaatttgacttcctcttttcctaattgaataccctgtatttccttatcctgccttattctcctggccagaacatccaacactgtgttgaataggagtggtgagagagggcatccctgtcttgtaccagtcttcaaagggaatgcttccaccttttgcccattcaacatgatatttgctgtgggtttgtcatagataactcttattatttggagatactccccatcaatacctaatttattgagagtttttagcatgaagtgttgttgaattttgtcaaaggtcttttctgcatctattgagataatcatgtggtttttgtcattggttctgtttatgtgctggattacattcattgatttgtgtacgttgaactagccttgcatcccagggatgaagcccacttggtcaaggtggataagctttttgatgtgctgctggattcagtttgccagtattttattgaggatttttgcatcgatgttcatcagggatattggtctacaattctcttttttttcttgtgtccctgccaggctttggtatcaggatgatgttggcctcataaaatgagttagggaggattccctctttttctgttgattggaatagtttcagaaggaagggtaccagctcctccttgtacctctggtagaattcagctgtgaatccatctggtcctggactttttttggttggtaagctattaattattgcctcaatttcagagcctgttattagtctattcagagattcaacttcttcctggtttagtcttgggagggtgtgtgtgtcgaggaatttatccatttcttcttgattttctagtttatttgcgtagaggtgtttatagtattctctgatggtagtttgtatttctctggaatcggtggtgatacccccttaatcatttttattccatctatttgattcgtctctcttttcttctttattagtcttgctagcagtctatcaattttgttggccttttcaaaaaaccagctcttggattcattgattttttgaagagttttttgtgtctctatttccttcagttctgctctgattttagttatttcttgccttctgctagcttttgaatgtgtttgctcttgcttctctagttcttttaattgtgacgttagggtgtcaattttagatgtttcctgctttctcttgtgggcatttagtgctataaatttccatctacacactgctttgaatgcgtcccagagattctggtatgttgtgtctttgttctcgttggtttcaaagaacatctttaattctgccttcatttcattatgtacccagtagtcattcagaagcaggttgttcagtttccatgtagttgaacagttttgagtgagtttcttaatcctgagttctagtttgattgcactgtggtctgagagatagtttgttataatttctgttcttttacatttgctgaggagtgctttacttccaactatgtggtcagttttggagttggtgtggtgtggtgctgaaaagaatgtacattctgttgatttggggtgcagagttctgtagatgtctattaggtcctcttcgtgcagagctgagttcaattcttggatatccttgttaactttttctcgttgatctgtctaatgttgacggtggtttgttaaagtgtcccattattattgtgtgggagtctaagtctctttgtaggtatctaaggacttgctttatgaatctgggtgcatgtatatttaggatagttagctcttcttgttgaattgatccctttaccattatgtaatggccttgtctcttttgatctttgctggtttaaagtctgttttatcagagactaggattgtaatccctgcctttttttgctttccatttgcttggtagatcttcctccatccctttattttgagcctatgtgtgtctctgcacatgagatgggtttcctgaatacagcacagtgatgggtcttgactcttgatccagtttgccagtctgtgtcttttaattctagcatttaacccatttacatttaaggttaatattgttatgtgtaaatttgatcctgtcattatgatgttagctgattgttttgctcgatagttgatgccgttttttcctagcctcagtggtctttatcatttggcatgtttttgcagtggctggtaccagttgttcctttccatgtttagtgcttccttcaggagctcttttagggcaggcctggtggtgacaaaatctctcagcatttgcttgtctgtaaatgcttagagattttattttatttctccttcacttatgaagcttagtttggctggatatgaaattctgggttgaagattcttttctttaagaatgttgaatattggcccccactctcttctggcttgtagagtttctgccaagagatcagctgttagtctgatgggcttccctttgtgggtaacctgacctttctctcttgctgcacttaacattttttccttcatttcaactttggtgaatctaacaattatgtgtcttggagttgctcttcttgaggagtatctttatggcattctctgtatgtcttgaatttgaatgtcagcctgccttgctagattggggaagctctcctggatgataacctgcagagtgttttccaacttgtttccattctccccgtcactttcaggtacaccaatcagatgtagatttggtcttttcacatagtcccatatttcttggaggctttgttcatttctttttattcttttttgtctaaacttctcttcttgcttcatttcattcatttcatcttccatcactgataccgtttcttccagttgatcaaatcagctactgaggcttgtgcattcatcacatagttctcgtgccatggttttcagctccatcaggtcctttaaggacttctctgcattggttatctagttagccattcatctaatcttttttcaaggtttttaccttctttgccttcggttcgaacttcctcctttagtccggagtagtttgatcgtctgaagccttcttctctgaactcgtcaaagtcattctccatccagctttgttccattgctggtgaggagctgcatttctttggaggaggagaggcattctgattttataatttccagtttttctgctcttttttcccccatctttgtggttttatctacctttggtttttgatgatggcgatgtagaaatgggttttggtgtggatgtcctttccgtttgttagttttccttctaacagtcaggaccctcagctgcaggtctgttcgagtttgctggaggtccactgcagatcctgtttgcctgggtatcagcagcggacactgcagaacagtggatattagtgaacagcaaatgttgctgtctgattgttcctctggaagttttctcagaggagtacccggctgtgtgaggtgtcagtctgcccctactggggggtgcctcccagtcaggctactcggggatcaggacccacttgagtagcagtctgtccattctcagatctcaagctgcgtgctgggagaaccactactcttttcaaagctgtcagacagggacatttaagactgcagaggtttctgctgccttttgtttggttatgccctgcccccagaggtggatctacagaggcaggcaggcgtccttgagctgcggtgggctccacccagttcaagcttctgggccgctttgtttacctattcaatccatgggaatggcaggcgcccctcccccagtctcactgctgccttgcagttcgatctcagactgctgtgctagcaatgagcgaggctccgtgggcgtaggaccctctgagccaggtgcgcgatataatctcctggtgtgccgtttgctaaaaccttttggaaaagcgcagtattagggtgggagtaacccaattttccaggtgccatctgttacccctatccttggctagggaagggaattcacagaccccttgtgcttccctgatgaggcaatgcctcgccatgctttggctcacacgtggtgcgctgcacccactgtcctgcacccactgtctgacagtcccctgggagatgaacccagtacctcagttggaaatgcagaaatcattcctcatctgtgtcattcacactgggagctgtagactggagctgttcttattcggccatcttggcttcttcccacagtaatttatatattcaaagctattcccatcaaactaccattgacattcctcacagagctagaaaaaaactactttaaatttaatatggaaccaaaaaagagcccatatagccaagacaaccctaagcaaaaagaacaaagctggaggcatcacgctacctgatgtcaaactatactattaggctacataaccaaaacagaatggtactggtaccaaaaagatgtattgaccaatggaacagaacatacgcctcagaaataacattacacatctacaaccatctgatctttgacaaacctgacaaaaacaagcaattaggggaaggattccctatttaataaatggtgttttggaaaaactggttagccatatggagaaaactgcaactggatcccttccttacaccttacagtaaccaaaccagcatagtactggaacaaaagcagacatatagaccaatggaacagaacagaggcctcagaaataacaccacacatctacagccatctgatcttcgacaaaactgacaaaaacaagcaatgggaaaggattccctatttaataaatggtgctggggaaactggctggccatatgcaaaaaacagaaactatacccctgccttacaccttatacaaaaattaactcaagatggattaaagacttaaatgtaaaacccaaaaccataaaaaccctggaagaaaacctaagcaacatcattcaggacataggcgtgggcaaaaatttcatgacacaaacaccaaaagcaattacaataaaagccaaagttgacaaatgggatcaaattaaactaaagagcttctgcacagcaaaataaaccatcatcagagggcacaggcaacctacagaatgagggaacatttttgcaatctatccctctgacaaaggtccaatatccagaatctacaagtaacttaaacaaatttacaagaaaaaaaacaccaccaccaaaaagtgggcaaaggatataaacagacacttctcaaaaaaatacatttatgcagccaacacacttatgagaaaagctcaatatcactgatcattagagaaatgcaaatcaaaaccacaatgagatacaatctcatgccagtcagaatggagatgattaaaaagtcaaaaaacaacagatgctggtgaaactggggagatataggaaaacttttacatttttggtgggaatgtaagttagttcaaccatcatggaaaacaatgtggtgattcctcaaagatctagaaccagaaacaccatttgacctagcaatctcattacttgatatatacccaaaggcatataaatcattctactgtaaagacacatgcacatgtatgtctattgcagcactatctacaatatcaaagatatggaaccaacacaaatactcatcaatgatagactggataaagaaaatatgatgcatgtacagtatggcggcatactatgtgccataaaaaggaatgagattatgtctgttgcagggacatggatgaaactcaaagccattatcctcagcaaactaacacaggaacagaaaaccaaacaccacatgttctcactcataagagggagtcgaaaaatgagaatacatggactcagggaggggagcaaaacacaccggggccattcggggcatggaatgggagtgagagtatcaggacaaatagctaatacatgagggacttaaaatctagatgacagattgataggtgctgcaaaccaccatggcacacatatacctatgtaacaaacccacacattctgcgtatgtatcctggaacttaaaataaaactgtttttaaaaaatgtgaaccgaccagatgtggtggctcaagcctgtaatcccagcactttgggtggctgaggagggcagatcacgaggtcaagagatcgagaccatactcctggccaacatagtgaaaccctgtctctactaaaaatacaaaaattagctaggcatggtggtgtgcacctgtagtcccagctcctcaggagtttgaggcaggagaatcacttgaacctgggagacggaggtttcagtgagctgagatagtgccactgcactccagcctggtgacagagcgagactccatctcaaagatcacaacaaaaaagtgaacctaccagtactcgcattaaatttaaacagaccaaagtagacaaaataatgatctttgaaaacttttatttaaggagataaaacatttgaagtacacaattaaatgtaacctatgtgttatatgtgtgtgtgtgtatatatattgcacatatatctgtatatatgtatccatacatatatctctggttgtgtgtgtgtagacagaaaataaacatttttaaaagagtttatggagactgggtgtggtggctcacacctgtaattccaacactttgggaggccaaggtgggaggatcacatttggtcaggagttcgagaccagcctggccaacatggtgaaatcctgtgtccactagtaatacaaaacttagccagatgtgggggatgagcacctgtagtcccagacacttgggaggttgaggcatgagaatcacttgaacccaggagacgtaagttgcagtcagccaagatcgctccgtcatactcaagcctgggtgacagagagagactctgtctcaaaaaaaaaaaaaaaaaaagattatatgtatgaaatagttactggaattgaccatatgctgggccataaaaaaatgctaagtaaattgtataagattcaaattcataaaaatagtatgttctagtcatactagaatcaaattataaatcatccagaaaatgataactaaaaacaatctctgattctttaaaataaaaaatattttttaaaaaattcagtcaaaaaattctaattataaatatgcctttctctcatcaactattcttccaagtatacatccaacaaaaatgaataagtatgtaaatctaataaaatatataaatatcttaataccataattattagtaagatcaaaaaatgttaacaacacaaatatccattaatcataaatgaagagatctgtggtctttgtcaatcaactgaatactctacaccagtgcaattaaacaactgctaaacaaaaacagaaatgaataagtattatttaattcaattatataaacataatctatgatattagaattcaggttaatacttactttgaggataaaagtagtgactaaaatggcccaggaggaaagagttcttctatgcagtttgtgataaattggtgttttcacttttggataatgtttaagataatcttaaaattttgagatagtcacatagcgtatatacatttatcctatgtatttcatacttaaacttttattgtggaagatatataacatattttaaagttccagaaatcaaatagcaaaataagaacgaagcagtagagaaatcctatgggtgcctatagtgtggccaggaaaacactctctaagaggtatcatttaaggggaagcctcaaaaagaaatagaagcaaacctcaagtagttgcaagaaagaatgctcaatgagagagacacggggaacaaaaggtgtaaacgggctaaggtgagaacaccttcaggagtttaaagaacaacagaaggccattgtctctagaagaaagtgtacagggggaaagggttaggacaagtaggtgtctgtgtagcgttctctagaccacagacagatttggattttattcttaatgtaacaagaaaccagatggaaatcttgacaggcaactgtcataatttgacattaatttagaaaatgttctggctactagtaggaaaatacggtagaagacagaagattagagtagaagctgagaaactagttaggagacatttcaccagttgtgacacaattcttctttaaatatgtcaaaatgggctgggcacagtggttcatgtctgtagggccaacactttgggagacagaggcagaatgattagaccacaggagttccagatgagtatgggcactacagcaagacctcatatctaaaaatgtaaaaaataaaaaatatagccaggcatggtggtgcgcagctgtagccccagcttctcatgaggctgaggtggaggattacctgagaccgaaacggaaaggctgcactgagccatgatcatgtgactgcactcagcctgtgtaatggagcaaaagcctgtctcaaaaaaaaaaaaaactaaaaatatcaaaatgatcattcccagattctatttccactatcttacttatagcacttagaatggctcacaatattttctgctctagaaaaacattaactttcccaccgaaaattccatttttcatttttaaaggtatttgtcagtgataaaactccaatttaaaaaccaaactttctgtaatgacatacattaaaacattaatattttatgtcaattcaatgacacttactttgaatcacttgtgtggcgcttttcaaagaccatccatagacttgatatgcttaagcaataaatttacttttaatgttgatatctttatatttatccttcagctataaagagaatatcatgaaattatcaagaattcatcatgatcaaccggtgaagcccctggatcgagcagtcttctggattgagtttgtcatgcgccataaaggagccaagcaccttcgggtcgcagcccacaacctcacctggatccagtaccactctttggatgtgatagcattcctgctggcctgcgtggcaactatgatatttatgatcacaaaatgttgcctgttttgtttccgaaagcttgccaaaacaggaaagaagaagaaaagggattagttatatcaaaagcctgaagtggaatgaccaaaagatgggactcctcctttattccagcatggagggttttaaatggaggatttcctttttcctgcgacaaaacgtcttttcacaacttaccctgttaagtcaaaatttattttccaggaatttaatatgtactttagttggaattattctatgtcaatgatttttaagctatgaaaaataataatataaaaccttatgggcttatattgaaatttattattctaatccaaaagttaccccacacaaaagttactgagcttccttatgtttcacacattgtatttgaacacaaaacattaacaactccactcatagtatcaacattgttttgcaaatactcagaatattttggcttcattttgagcagaatttttgtttttaattttgccaatgaaatcttcaataattaaa

SEQ ID NO: 8 - protein, 530 aa, Homo sapiens
MSLKWMSVFLLMQLSCYFSSGSCGKVLVWPTEYSHWINMKTILEELVQRGHEVIVLTSSASILVNASKSSAIKLEVYPTSLTKNDLEDFFMKMFDRWTYSISKNTFWSYFSQLQELCWEYSDYNIKLCEDAVLNKKLMRKLQESKFDVLLADAVNPCGELLAELLNIPFLYSLRFSVGYTVEKNGGGFLFPPSYVPVVMSELSDQMIFMERIKNMIYMLYFDFWFQAYDLKKWDQFYSEVLGRPTTLFETMGKAEMWLIRTYWDFEFPRPFLPNVDFVGGLHCKPAKPLPKEMEEFVQSSGENGIVVFSLGSMISNMSEESANMIASALAQIPQKVLWRFDGKKPNTLGSNTRLYKWLPQNDLLGHPKTKAFITHGGTNGIYEAIYHGIPMVGIPLFADQHDNIAHMKAKGAALSVDIRTMSSRDLLNALKSVINDPIYKENIMKLSRIHHDQPVKPLDRAVFWIEFVMRHKGAKHLRVAAHNLTWIQYHSLDVIAFLLACVATMIFMITKCCLFCFRKLAKTGKKKKRD

SEQ ID NO: 9 - DNA, 3742 bp, Homo sapiens
tttccacatgtgaggggctgaggggctcaaggaggggagcatctccactgggtggaggctgggggtcccagcaggaagtggtgagacaaagggcgctggctggcagggagacagcacaggaaggtcctagagcttcctcagtgcagctggactctcctggagaccttcacacaccctgacatctgggccttgcccgacgagggtgctttcactggtctgcaccatggcccaggccctgggattttgaacagctccgcaggtgaatgaaaggtgaggccaggctggggaaccaccgcattagagcccgacctggttttcagccccagccccgccactgagtggctttgtgagtgcgggcaagtcactcagcctccctaggcctcagtgacttccctgaaagcaagaattccactttcttgctgttgtgatggtggtaagggaacgggcctggctctggcccctgacgcaggaacatggagctgatccaggacacctcccgcccgccactggagtacgtgaagggggtcccgctcatcaagtactttgcagaggcactggggcccctgcagagcttccaggcccggcctgatgacctgctcatcagcacctaccccaagtccggtaagtgaggagggccacccaccctctcccaggtggcagtccccaccttggccagcgaggtcgtgccctcagcctgctcacctcccatctccctccctctccaggcactacctgggtaagccagattctggacatgatctaccagggtggtgacctggagaagtgtcaccgagctcccatcttcatgcgggtgcccttccttgagttcaaagccccagggattccctcaggtgtgtgagtgtgtcctgggtgcaaggggagtggaggaagacagggctggggcttcagctcaccagaccttccctgacccactgctcagggatggagactctgaaagacacaccggccccacgactcctgaagacacacctgcccctggctctgctcccccagactctgttggatcagaaggtcaaggtgaggcagggcacagtgtttcacatccataatcccagcactttgggaggctgaggcaggcagatcacctgaggttgggagtttgagagcaccctgagcaacatagaagaaccttgtctctactaaaaatacaaaattagccgggtgtggtggcgggtgcctgtaatcccagctactccgaagcctgagacaggagaatcacttgaacccgggagaaggaggttgtggtgagccagagatcccaccattgcattccagcctgagcaacaagagcaaaactcacaaaaataaataaataaataaatatataaataaaaataaaactgtggcacctgtggtggctcactgctgtaatgccagcactttgggaggccaaattgggtggatcacttgagctcaggagttacagaccagcccgggaaacatggggagtctccatctctataaaaatgcaaaatatcagcagggcatggtggcatagcgctgtagttccagctactggaaagtctgaggttggaggattgcttgagcctggaaggtcaaggttgcagtgagttattatcactccagtgcactccaacttgggcgacagaaaaaaagaaagaccacggtccttttttttttttttttttttttgagactctctgtctcaaaaataaataaataaaataaaataaaataaaataaatcccacaataaaaagaaaaagcaaaggtccaggtgtggggcatgtgaatccagggaaggaggccctggctcagcccagctttggtcctgttcttctgggaaagtcgcctcacttcctccagccttgtctcatcttctgcggcggggactgtctgcctcttgctctgatgaccaagaacgtaagactcttcagtgtagacctaagaaagctagagggtgggtcctcacaggcccacaaaatttggtggcggtgggatcacggctggtggagcatgccttgctccagattggggtgtgacgcattgatgcagattatattactatagaatatgatggtctcagggaccaggcaggactttggcttctgagcagggttcagctcctgacttggccctaccggtgccgtgagatctcaaacaagtcagcctctaagcctcaggttcctcctttgccaatccaagagatgagctggcctgggacaggctgtgtggtgatggtgctggggttgagtcttctgcccctgcaggtggtctatgttgcccgcaacgcaaaggatgtggcagtttcctactaccacttctaccacatggccaaggtgcaccctgagcctgggacctgggacagcttcctggagaagttcatggtcggagaaggtgggtttgatgggaggaaggaaagtgtggagctaaggggtggtggctacaacgcacagcaaccctgtgttggcaccccttgcctgcttctccagtgtcctacggatcctggtaccagcacgtgcaggagtggtgggagctgagccgcacccaccctgttctctacctcttctatgaagacatgaaggaggtgagaccacgtgcgatgcttccctccatgtgactcctgggggcaggcacctcacagggacccgccaaggccacccagccccctccctgggcagcccccacagcaggaccagattccccatcctgccttcttggcccaggcctccccactaaggccccacctggcagcgggccccacacagctctcatctctcgcacctgagtcagctgcatggggggccacggatcagaaacttagtcctattgctactccctgccaaagggtgtgctacccagggccacagtcacagaagaagaccatcacggtcctcacccataggagccaagcccagctcatgatgggatcacagggcagacagcaattctttttacccccgggactggggccctgggggttgaggagttggctctgcagggtttctaggagaagtggccagatcgcctctgaggttagagaaggggaccccttttacttttcctgaatcagtaatccgagcctccactgaggggccctctgctgctcagaacccgaaaagggagattcaaaagatcctggagtttgtggggcgctccctgccagaggagaccgtggacttcgtggttcagcacacgtcgttcaaggagatgaagaagaaccctatgaccaactacaccaccgtcccccaggagttcatggaccacagcatctcccccttcatgaggaaaggtgggtgctggccagcacgggggtttggggcgggtgggagcagcagctgcagcctccccataggcacttggggcctcccctgggatgagactccagctttgctccctgccttcctcccccaggcatggctggggactggaagaccaccttcaccgtggcgcagaatgagcgcttcgatgcggactatgcggagaagatggcaggctgcagcctcagcttccgctctgagctgtgagaggggctcctggagtcactgcagagggagtgtgcgaatcaaacctgaccaagcggctcaagaataaaatatgaattgagggcccgggacggtaggtcatgtctgtaatcccagcaatttggaggctgaggtgggaggatcatttgagcccaggagttcgagaccaacctgggcaacatagtgagattctgttaaaaaaataaaataaaataaaaccaatttttaaaaagagaa

SEQ ID NO: 10 - protein, 295 aa, Homo sapiens
MELIQDTSRPPLEYVKGVPLIKYFAEALGPLQSFQARPDDLLISTYPKSGTTWVSQILDMIYQGGDLEKCHRAPIFMRVPFLEFKAPGIPSGMETLKDTPAPRLLKTHLPLALLPQTLLDQKVKVVYVARNAKDVAVSYYHFYHMAKVHPEPGTWDSFLEKFMVGEVSYGSWYQHVQEWWELSRTHPVLYLFYEDMKENPKREIQKILEFVGRSLPEETVDFVVQHTSFKEMKKNPMTNYTTVPQEFMDHSISPFMRKGMAGDWKTTFTVAQNERFDADYAEKMAGCSLSFRSEL

SEQ ID NO: 11 - DNA, 19149 bp, Homo sapiens
  allele (11397)..(11397): C
gatgggattggggttttcccctcccatgtgctcaagactggcgctaaaagttttgagcttctcaaaagtctagagccaccgtccagggagcaggtagctgctgggctccggggacactttgcgttcgggctgggagcgtgctttccacgacggtgacacgcttccctggattgggtaagctcctgactgaacttgatgagtcctctctgagtcacgggctctcggctccgtgtattttcagctcgggaaaatcgctggggctgggggtggggcagtggggacttagcgagtttgggggtgagtgggatggaagcttggctagagggatcatcataggagttgcattgttgggagacctgggtgtagatgatggggatgttaggaccatccgaactcaaagttgaacgcctaggcagaggagtggagctttggggaaccttgagccggcctaaagcgtacttctttgcacatccacccggtgctgggcgtagggaatccctgaaataaaagatgcacaaagcattgaggtctgagacttttggatctcgaaacattgagaactcatagctgtatattttagagcccatggcatcctagtgaaaactggggctccattccgaaatgatcatttgggggtgatccggggagcccaagctgctaaggtcccacaacttccggacctttgtccttcctggagcgatctttccaggcagcccccggctccgctagatggagaaaatccaattgaaggctgtcagtcgtggaagtgagaagtgctaaaccaggggtttgcccgccaggccgaggaggaccgtcgcaatctgagaggcccggcagccctgttattgtttggctccacatttacatttctgcctcttgcagcagcatttccggtttctttttgccggagcagctcactattcacccgatgagaggggaggagagagagagaaaatgtcctttaggccggttcctcttacttggcagagggaggctgctattctccgcctgcatttctttttctggattacttagttatggcctttgcaaaggcaggggtatttgttttgatgcaaacctcaatccctccccttctttgaatggtgtgccccaccccgcgggtcgcctgcaacctaggcggacgctaccatggcgtgagacagggagggaaagaagtgtgcagaaggcaagcccggaggtattttcaagaatgagtatatctcatcttcccggaggaaaaaaaaaaagaatgggtacgtctgagaatcaaattttgaaagagtgcaatgatgggtcgtttgataatttgtcggaaaaacaatctacctgttatctagctttgggctaggccattccagttccagacgcaggctgaacgtcgtgaagcggaaggggcgggcccgcaggcgtccgtgtggtcctccgtgcagccctccggcccgagccggttcttcctggtaggaggcggaactcgaattcatttctcccgctgccccatctcttagctcgcggttgtttcattccgcagtttcttcccatgcacctgccgcgtaccggccactttgtgccgtacttacgtcatctttttcctaaatcgaggtggcatttacacacagcgccagtgcacacagcaagtgcacaggaagatgagttttggcccctaaccgctccgtgatgcctaccaagtcacagacccttttcatcgtcccagaaacgtttcatcacgtctcttcccagtcgattcccgaccccacctttattttgatctccataaccattttgcctgttggagaacttcatatagaatggaatcaggctgggcgctgtggctcacgcctgcactttgggaggccgaggcgggcggattacttgaggataggagttccagaccagcgtggccaacgtggtgaatccccgtctctactaaaaaatacaaaaattagctgggcgtggtgggtgcctgtaatcccagctattcgggagggtgaggcaggagaatcgcttgaacccgggaggcagaggttgcagtgagccaagatcgtgccactacactccagcctgggcgacaagaacgaaactccgtctcaaaaaaaaggggggaatcatacattatgtgctcatttttgtcgggcttctgtccttcaatgtactgtctgacattcgttcatgttgtatatatcagtattttgctccttttcatttagtatagtccatcgattgtatatccgtccttttgatggccttttgagttgtttcccatttgcggttatgaaataaagctgctataaacattcttgtacaattctttttgtgatcatatgttttcgtgtttcttggagaaatacttaggaggggaattgcgagtttggaagtaaaaagtagctgtattttgaactttttcagaagctctgagttttccagagcggttgtaccattttacactccaactagcaaggtatgggagttattatggttgtgccacagccttccggacattaggtattgtcagtctttctaatgtggtatatccttgtggttgtaatttacagttctctattgactaaggatgttcagcattttttcatgtgcctattggccattcgtattttgtttgtaaagtagctcttcgagtcttttacctgttattttggttttttgtttgtttttattgttcagttgtgggactgctttatacattctggatacaagtcctttatcagatccatgtgtcgtgaatgttttcttctgatctgttgcttgcctatttgtttgctttacagagtttacagtatcttaagaggagtggatttatcttttttatgttcagtatttgccttgtcctgtttaggacatctttttttttttttttaaccccagggtcatgaagatattatcttacattttcttttaggacctttatggttgtaagttttacagtaaggtccttgagccattaattaattcttaaaattaattgtttatggtgtgaggtgtaggagtcagtctctggtatctttcctgtatggaaatccagttattctgtctccacttgttgaaataggcttcctttctctactgaatgcttttaattttaattattttacagttggagtatagggctaccattttagtgctattttctttttttctttgttaatttttgagacagggactcacactgttgcccaggctagagtacaatggcacaatcaaggcttactgcagcctcgaacccctgggctcaagcagtcctctagcagcctcacgagtagctgggattactccaccacacccagctaactattttatttttttgtattgacaggatctcactatgttgcccaggctggtctcaaactgctggcctcaagctttcatcccatctcggcctcccaaagtgctgggattacaggtgtgagccaccatgcctgacctcttagtgctattttctatttatctcctctgttctctgctctctttaaacgttggaggaagaaacagtacccatcttacacaaactcttcagaaaacagaggaacagactgggcgcggtggctcatacctgtaatctcagcactttggtacgctgaggcaggggatcatttgaggtcgggagttcgagaccagcctggccaacacggcgaaaccccatctctactaaaaatacaaaaagtagctaggcgtggtgacacatacctgtaatgccagttactcaggaggctgaggcacaagaatcccttgaacctgggaagcggaggttgcagtgagccgagattgcgccactgcactccagcctgggcaacagagtgagaccctgtctcagaaaaaaaaagaaagaaagaaaaaatagaggaatatttcccaacttgttttcgaagccagcataatcctggtaccaaaaccaaacaaggacattataagaaaagaaaatatagaccaatattcctgttagcatagacatgcaacagctaaccaattttagcaaaccaaacctggtaatatagaaaaaaggataaataggccagtcgcggtggctcacgcctgtaatcccagcactttgggaggctgaggcaggcagatcacttgaggtcaggagtttgagaccagcctgaccaacatggtgaaaccccgtttctaataaaaatacaaaaatcaggctgggcacggtggctcacgcctgtaatcccagcactttgggaggccgaggtgggcagatcacgaggtcaggagttcaagaccagcctgaccaatgtggtgaaacgccatctctactaaaaatacaaaaatcagccggtgtggtggcacctgcctgtaatcccagctactcaggaggctgaggcagaattgcttgaacccgggaggcagaggttgcagtgagccaagatcgtgccactgcactccagcctgggcgacagagcaagacttcatctcaaaaaaaaaaaaaaattagctgggcatggtggtgggcacctgaaatcccagctactcgggagtctgaggcaggagaatcgcttgaacccaggaggcagaagttgcactgagctgggatcacaccattgcactccagcctgggcaacagagtgagactccatctcaaaaaaagaaaaagaaaaaggataaatacattctaaccaaataatgtttatctcatgattgtagctgattcaacattcaaaaattggcctggtgcagtagctcaggcctgtaatcccaacattttaggaggctgaggcaggaagatctcttgagcccaggatttcaagaccagcctgggcaacatagtcagactggtctttactggggggaaaaaaatcagtctgtgtaattcaccacattaacaaagggaaacataaaaaccctatgatcatttcaacagatgtagcaaaagcagttaatgatattcaacacatatgcatgattacaaaccaaccaacctcctagcaaactagggaaaggaaacttaacctagtttgataacagggcgtccacagtcggagttccactagcagcatacataatggtagaaaactcagtgctgccgggcgcggtggctcacgcctgtaatgccagcactttgggaggcctaggcgggcggatcacgaggtcaggagatcgagactgtcctgactagcatgctgaaaccccgtctctactaaaaatacaaaaacaaaaaattagccgggcatggtggcgggcgcctatagtcccagctactcgggaggctgaggcgagagaatggcgtgaacccgggaggcggagcttgcagagcctagatcgtgccactgcactccagcctgggtgacagagtgagacttcgtctcaaaaaaaaaaaaaaaaaaaaaagaaaagaaaactcaacgctttttcctctaagatcaggaactagaaaaggatttgactctcacaacgttgataccatactggaggttttaaccaggcaagaaaaagaaataatgagggccgggtgcggtggctcaggcctgtaatcccagcactttgggaagccgagacgggtggatcacgaggtcaggagatcgagaccatcctggctaacacggtgaaaccctgtctctactaaatatacaaaaaattagccgggcgtagtggcgggcgcctgtagtcccagctactcgggaggctgaggcaggagaatggcgtgaactcagggggcggagcttgcagtgagctgagatcgagccactgcactccagcctgggcgacagagcaagactgtgtctcaaaaaaaaaaaaagaaaaagaaataatgattagtggcccgatgtctcacgcctataatcccagcactttgggaggccgaggtgggcagatcacctgaggtctggagttggagaccagcctgacaaagatggtgaaacctcgtctctattaaaatattaaaaaaatagccaggcgttggccgggtacagtggctcatgcctgtaatcccagcactttgggaggccgaggtgggtggatcacctgaggtcaggagttcaacaccagcctggccaacatggtgaaaccccatctctactaaaaatacaaaaattagccgggcgtagtggcgggcgcctgtaatcccagctacttgggaggcttaggcaggagaatcgcttgaacctgggaggcggaggttgtagtgagccgagattgcaccattgcactccagcctgggtgacaaaagcaaaaactccgtctcaaaaaaaaaagaattagccaggggtagtggtgaacgcctgtagtcccagctactcaggaggcagaggcaggagaatcacttgaacccaggaggcagaggttgcagtgagccgagattgtcccattgcactccagcctaggcgacaagagcaaaattccatgtcaaaaaaaaaaaaaaaaaaggaaagaaaaaaaataacgattagaaaggaagaaataaaacacattcacagccagtatgattctatacatacatgtcctaatggggccaggcgtggtggctcatgcctgtaatcctagcacttttaggaggctgaggcaggtggcttccctgggaccagcctggccaacatggtgaaaccccaactctaataaaaatacaaaaaatcagccaggcgtggtgacgggcacctctaatcccagctactcaggaggctgaggcaggagaattgcttggacctgggaggcagaggttgcagtgagccgagatcgcgctattgcactccagcctgggcaacaagagtgaaactccggcagggtgtggtggcttacgcctgtaatcccagcacttcgggaggctgaggcaggccgatcacctgaggtcaggagtttgagaccaacctaacatggtgaaaccccgtctctactaaaaatacaagaattagctgggtgtagtggtgggcgcctgtaatcccagctacttgggaggctgagacagaagaattgcttgaacccaggaggtggaggttgcagtgagctgagatcatgccattgcacaccacgccgggcaacagagcgagattccgtctcaaaaaaaaaaaaaaagagtgaaactctatctcaaaaaaaaaaaaaagtcctaatggaaaatccataaaaagctaccaaaactaataaataaatatagcagggttgcaggttacagggcaatatagttatccctctatctgtaggggcttggttctgggactcctcacacaccaaacccacagatgtctaagtcccatatataagacggtatagtatttggatttaacctacacatatcctcccatatagtttaaattatctctagattacttacattacccccatacaatgaaaatgctaatgtacatgcaagtatgtatgtaagtacttgtactatattgtttagggaatcactggacatataggccttcaagactgataccagcagccactgttaagattctggtcaggcctgcccctgtttggggtctcagttgatctcattgccttcccacccagccaagggcacctgcatttctcttggctccctggccatttggaaggcctagttcagcctggcacatttgtatcctggcccactgatgctggtacccctgggaaggtcctgctctgaaaaacacggagattttagttgctactgaagatttgagagataaagacagggagacctgtctgtagacctgtgtccctccaagtgggattgagactttgggccccccatttcaggacagcacctcctggcctgttgactgaatagatccctgaaggaggtgtacttgcattaatggagtgggggtgggagcagtaccacagatccgcactaacaatcacacagttctctctagaataataatatagaacaagtgaaatagaacaattgcagaaagagctaacctttgttgagctcttactgtgtgcccagcactttcctcaactctacatttcccataatacacagagtactaggtaggccaggcttggtggctcacgcctgtaatcccagcactttaggaggccaaggggggtggatcacctgaggtcgggagttcaagaccagcctgaccaacatggtgaaaccccgtctctactagaagtacaaaattagccaggtgtggtggcacatgcttgtagtcctagctactcagcaggctgaggcaggagaatcatttgaatccgggaggaggttgcagtaagcggagatagtgccactgtactccagcctgggcaataagagctgagactccgtctcaaaataaaataaaataaaataaaataaaataaaataaaataaaaaaagaaaagagcctgccattaaaggagctgtttggtaggggatgttttgtcagtgcaaacaacagaaaagtgggctgggcacagtggttcatgcctgtaatcccagcactttgggaggccaaggcgggcggatcacctgaagttgggagttcaagaccagcctgaccaatatggagaaaccccgtctctactaaaaatacaaaattagccgggcgcagtggcgcatgcctgtaatcccagctactcgggaggctgaggcaggagaatcgcttgaacctgggaggcagaggttgcggtgagccgagatcgcaccattgcactccagcctggacgagagcaaaactctgtctcaaaaaaaaaaaaaaacagaaaagtgtaacaaacacttacagtaggcatgtttcttagcaaatctgatgacaaatttggcataaagaaagagagcatccctgaaaaaaaaaaaaagaaaaagaaagagagcatcctgcctgggcaacatagtgaaaccctgcctctacaaaaaaactcaaaaattggccgggtgcagtggctcacacctgtaatcccagcactttgggagtcggaggcgggaggatcacctgaggtcaggagttcgaaaccagcctggccaacatggcaaaaccccatctctactaaaaatacaaaaaattaatcaggcgcattggtgggcgcctgtaatcccagctactcaggaagttgaggcaagaggatcgcttgaatctgggaggtggaggttacagtgagtcgagatcacaccactgcactctagcctgggtgacagggcgagactccgtctccaaaaaaaaaaagaaaaagaaaaagactaaaaaattagccaggcaggcctctgtggtcccagctacttgggaggctgaggcaggagaatcactgagcccaggagtccgaggctgtagtgagccatgattgcaccactgtaccctagcttgggcaacaaagcaagaccctgcctcaaaagaaaaaagaaagaaagaaagaacatggcgggccaggcacagtggctcacacctgtaatcccagcgctttgagaggccgaggcaggtggatcacaaggtcaggagttccacaccagcctggccaacatggtgaaaccctgtctctactaaaaatacaaaaaatcagccaggcatggtggcaggggcctgtaatcccagctactcgggaggctgaggcaggagaattgcttgaaaccagaaggcagaggttgcagtgagcctagactgcaccactgcactccagcctgggcgaaaagagccaaactccatctcaaaaaacaaacaaaaaaacaaaacaaaagaaaacatggcaaagcctttgaaagcttgtctgggagaaggtgcgatgatagttgcataacttcgtgcaagatgctggtccacacaggggctgccccttgctctttctcgctctcttaacctctcatataacaggcttgtgtgttattcacatttattgagcccaagcaggtgcaaggcattgtgatctaatactttggtcagcaagacaacaagatagatcactgccctgcccttaggaagtgtatatgctattagaggaaacagataaaataaacaaggaaaagtatcagacaatgtaagtgctatgagaatgcaaatgaggtgatgtgaattaaaataggatgacttaaagtctgcacgggaaggagcctacccccatgttcctggctagccaaggaaccaccagttgattagcagagaagggcagccagtctagctagagcttttggggaagagggagtggttgttaagagatgagattaaagaagccgagacgggccattcgtgaggggtttgtaatgcagggctgaggagtgtccgaagagaatgggcaggtgagcggtgagacagttgttcttccagaagctttgcagtgaaaggaatcaaagaaatggagccgtgtatcaggtggggaagggtgggggccaagggggtgtccttccccatacagagattgcaggctgagaatgactatatccttgttaacaggaggtgggagcagggcacggtagctcacacctgtaatcttggcactttaggaggctgaggcgggccgatcacctgaagtaaggagttcgagaccagcctggccaacatgcaaagccctgtctctactaaaaatacaaaaattagctgggtgtggtggtactcgcctgtaatcccagctactcgggagactgaggcaggagaatggcttgaacccggaaggtagaggttgcagtgagctgagatcatgccactgtgctccagcctaggtgacagagagagactccatctcaaaaaaaaaaaaaaaatacaggaagggagttgggaatagggtgcacatttaggaagtcttggggatttagtggtgggaaggttggaagtccctctctgattgtcttttcctcaaagaagtgcatggctggtgaggggtggggcaggagtgcttgggttgtggtgaaacattggaagagagaatgtgaagcagccattcttttcctgctccacaggaagccgagctgtctcagacactggcatggtgttgggggagggggttccttctctgcaggcccaggtgacccagggttggaagtgtctcatgctggatccccacttttcctcttgcagcagccagactgccttccgggtcactgccatggaggagccgcagtcagatcctagcgtcgagccccctctgagtcaggaaacattttcagacctatggaaactgtgagtggatccattggaagggcaggcccaccacccccaccccaaccccagccccctagcagagacctgtgggaagcgaaaattccatgggactgactttctgctcttgtctttcagacttcctgaaaacaacgttctggtaaggacaagggttgggctggggacctggagggctggggacctggagggctggggggctggggggctgaggacctggtcctctgactgctcttttcacccatctacagtcccccttgccgtcccaagcaatggatgatttgatgctgtccccggacgatattgaacaatggttcactgaagacccaggtccagatgaagctcccagaatgccagaggctgctccccgcgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacggtcagttgccctgaggggctggcttccatgagacttcaatgcctggccgtatccccctgcatttcttttgtttggaactttgggattcctcttcaccctttggcttcctgtcagtgtttttttatagtttacccacttaatgtgtgatctctgactcctgtcccaaagttgaatattccccccttgaatttgggcttttatccatcccatcacaccctcagcatctctcctggggatgcagaacttttctttttcttcatccacgtgtattccttggcttttgaaaataagctcctgaccaggcttggtggctcacacctgcaatcccagcactctcaaagaggccaaggcaggcagatcacctgagcccaggagttcaagaccagcctgggtaacatgatgaaacctcgtctctacaaaaaaatacaaaaaattagccaggcatggtggtgcacacctatagtcccagccacttaggaggctgaggtgggaagatcacttgaggccaggagatggaggctgcagtgagctgtgatcacaccactgtgctccagcctgagtgacagagcaagaccctatctcaaaaaaaaaaaaaaaaaagaaaagctcctgaggtgtagacgccaactctctctagctcgctagtgggttgcaggaggtgcttacgcatgtttgtttctttgctgccgtcttccagttgctttatctgttcacttgtgccctgactttcaactctgtctccttcctcttcctacagtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtgagcagctggggctggagagacgacagggctggttgcccagggtccccaggcctctgattcctcactgattgctcttaggtctggcccctcctcagcatcttatccgagtggaaggaaatttgcgtgtggagtatttggatgacagaaacacttttcgacatagtgtggtggtgccctatgagccgcctgaggtctggtttgcaactggggtctctgggaggaggggttaagggtggttgtcagtggccctccaggtgagcagtaggggggctttctcctgctgcttatttgacctccctataaccccatgagatgtgcaaagtaaatgggtttaactattgcacagttgaaaaaactgaagcttacagaggctaagggcctcccctgcttggctgggcgcagtggctcatgcctgtaatcccagcactttgggaggccaaggcaggcggatcacgaggttgggagatcgagaccatcctggctaacggtgaaaccccgtctctactgaaaaatacaaaaaaaaattagccgggcgtggtgctgggcacctgtagtcccagctactcgggaggctgaggaaggagaatggcgtgaacctgggcggtggagcttgcagtgagctgagatcacgccactgcactccagcctgggcgacagagcgagattccatctcaaaaaaaaaaaaaaaaggcctcccctgcttgccacaggtctccccaaggcgcactggcctcatcttgggcctgtgttatctcctaggttggctctgactgtaccaccatccactacaactacatgtgtaacagttcctgcatgggcggcatgaaccggaggcccatcctcaccatcatcacactggaagactccaggtcaggagccacttgccaccctgcacactggcctgctgtgccccagcctctgcttgcctctgacccctgggcccacctcttaccgatttcttccatactactacccatccacctctcatcacatccccggcggggaatctccttactgctcccactcagttttcttttctctggctttgggacctcttaacctgtggcttctcctccacctacctggagctggagcttaggctccagaaaggacaagggtggttgggagtagatggagcctggttttttaaatgggacaggtaggacctgatttccttactgcctcttgcttctcttttcctatcctgagtagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgaggtaagcaagcaggacaagaagcggtggaggagaccaagggtgcagttatgcctcagattcacttttatcacctttccttgcctctttcctagcactgcccaacaacaccagctcctctccccagccaaagaagaaaccactggatggagaatatttcacccttcaggtactaagtcttgggacctcttatcaagtggaaagtttccagtctaacactcaaaatgccgttttcttcttgactgttttacctgcaattggggcatttgccatcagggggcagtgatgcctcaaagacaatggctcctggttgtagctaactaacttcagaacaccaacttataccataatatatattttaaaggaccagaccagctttcaaaaagaaaattgttaaagagagcatgaaaatggttctatgactttgcctgatacagatgctacttgacttacgatggtgttacttcctgataaactcgtcgtaagttgaaaatattgtaagttgaaaatggatttaatacacctaatctaaggaacatcatagcttagcctagcctgctttttttttttttttttttggagacagagtctcactctgtcacccaggctggagtgcagtggcgggatctcggctcactgcaacctccgccttctgggttcaagcgattctcctgcctcagcccactgagtagctgggattacaggcacctgccccgacgcccagctaattttttgttatttatttatttttttttttagtagagatgaggtttcaccatgttggccaggctagtctcgaactcctgaccttgtgatctgcctgccttggcctcccaaagtgctgggattacaggcgtgagccaccgcacccggcctgcctagcctacttttattttatttttaatggagacagcatcttgctctgttgcccaggctggattacagtgatgtgatcatagctcattataccctcctgggctcaagcaatccccctaactctgcctccccagtagctaggaccacaggcatacaccaccatacccagctaatttttaaaattttttgtagatagatagagtctcactatgttgcccaggctggtctctagcctacttttttgagacaaggtcttgctctgtcacccaggctggatagagtgcagtagtgcagtcacagctcactgcagcctccacctcccaggctccatccatcctcccagctcagcctcccaagttgcttcaactacaggcctgcaccaccatgcctggctaatttttatttatttatttttattttattttattttatttttttgagactcagtctcactctgtcgcccaggctggagtgcagtggcatgatctcggctcactgcaacctctgcctcctgggttcaagtgattctcctgcctcagcctcccgaatagctaggactacaagcgcctgctaccacgcccagctaatttttgtatttttagtagagacagggtttcaccatgttggccaggctggtctcgaacttctgaccatgtgatccgcccgcctcggcctcccaaagtgctgggattacaggtgtgagccaccacgcccggctaatttttatttatttatttaaagacagagtctcactctgtcactcaggctagagtgcagtggcaccatctcagctcactgcagccttgacctccctgggctccggtgatttcaccctcccaagtagctaggactacaggcacatgccacgacacccagctaattttttattttctgtgaagtcaaggtcttgctacgttgcccatgctggtatcaaacccctgggctcaatcaatccttccacctcagcctccccaagtattggggttacaggcatgagctaccacactcagccctagcctacttgaaacgtgttcagagcatttaagttaccctacagttgggcaaagtcatctaacacaaagccctttttatagtaataaaatgttgtatatctcatgtgatttattgaatattgttactgaaagtgagaaacagcatggttgcatgaaaggaggcacagtcgagccaggcacagcctgggcgcagagcgagactcaaaaaaagaaaaggccaggcgcactggctcacgcctgtaatcccagcatttcgggaggctgaggcgggtggatcacctgaggtcaggagttcaagaccagcctagccaacatggtgaaaccccgtctctactaaaatacaaaaattaaccgggcgtgatggcaggtgcctgtaatcccagctacttgggaggctgaggcaggagaatcgcttgaaccaggaggcggaggttgcagggagccaagatggcgccactgcactccagcctgggcgatagagtgagactccgtctcagaaaaaaaagaaaagaaacgaggcacagtcgcatgcacatgtagtcccagttacttgagaggctaaggcaggaggatctcttgagcccaagagtttgagtccagcctgaacaacatagcaagacatcatctctaaaatttaaaaaagggccgggcacagtggctcacacctgtaatcccagcactttgggaggtggaggtgggtagatcacctgacgtcaggagttggaaaccagcctggctaacatggtgaagccccatctctactaaaaacacaaaaattagccaggtgtggtagcacacgcctgtagtcccagctactcgggaggctgaggcacaagaatcacttgaaccccagaggcggagattgcaatcagccaagattgcaccattgcactcccgcctgggcaacagagtgagaccccatctcaaaataaataaataaatatttttaaaagtcagctgtataggtacttgaagtgcagtttctactaaatgcatgttgcttttgtaccgtcataaagtcaaacaattgtaacttgaaccatcttttaactcaggtactgtgtatatacttacttctccccctcctctgttgctgcagatccgtgggcgtgagcgcttcgagatgttccgagagctgaatgaggccttggaactcaaggatgcccaggctgggaaggagccaggggggagcagggctcactccaggtgagtgacctcagccccttcctggccctactcccctgccttcctaggttggaaagccataggattccattctcatcctgccttcatggtcaaaggcagctgaccccatctcattgggtcccagccctgcacagacatttttttagtcttcctccggttgaatcctataaccacattcttgcctcagtgtatccacagaacatccaaacccagggacgagtgtggatacttctttgccattctccgcaactcccagcccagagctggagggtctcaaggaggggcctaataattgtgtaatactgaatacagccagagtttcaggtcatatactcagccctgccatgcaccggcaggtcctaggtgacccccgtcaaactcagtttccttatatataaaatggggtaagggggccgggcgcagtggctcacgaatcccacactctgggaggccaaggcgagtggatcacctgaggtcgggagtttgagcccagcctgaccaacatggagaaaccccatctctactaaaaatacaaaagtagccgggcgtggtgatgcatgcctgtaatcccagctacctactcgggaggctgaggcaggagaatcgcttgaacccgggaggcagaggttgcggtgagctgagatctcaccattacactccagcctgggcaacaagagtgaaactccgtctcaaaaaagataaataaagtaaaatggggtaagggaagattacgagactaatacacactaatactctgaggtgctcagtaaacatatttgcatggggtgtggccaccatcttgatttgaattcccgttgtcccagccttaggcccttcaaagcattggtcagggaaaaggggcacagaccctctcactcatgtgatgtcatctctcctccctgcttctgtctcctacagccacctgaagtccaaaaagggtcagtctacctcccgccataaaaaactcatgttcaagacagaagggcctgactcagactgacattctccacttcttgttccccactgacagcctcccacccccatctctccctcccctgccattttgggttttgggtctttgaacccttgcttgcaataggtgtgcgtcagaagcacccaggacttccatttgctttgtcccggggctccactgaacaagttggcctgcactggtgttttgttgtggggaggaggatggggagtaggacataccagcttagattttaaggtttttactgtgagggatgtttgggagatgtaagaaatgttcttgcagttaagggttagtttacaatcagccacattctaggtaggggcccacttcaccgtactaaccagggaagctgtccctcactgttgaattttctctaacttcaaggcccatatctgtgaaatgctggcatttgcacctacctcacagagtgcattgtgagggttaatgaaataatgtacatctggccttgaaaccaccttttattacatggggtctagaacttgacccccttgagggtgcttgttccctctccctgttggtcggtgggttggtagtttctacagttgggcagctggttaggtagagggagttgtcaagtctctgctggcccagccaaaccctgtctgacaacctcttggtgaaccttagtacctaaaaggaaatctcaccccatcccacaccctggaggatttcatctcttgtatatgatgatctggatccaccaagacttgttttatgctcagggtcaatttcttttttcttttttttttttttttttctttttctttgagactgggtctcgctttgttgcccaggctggagtggagtggcgtgatcttggcttactgcagcctttgcctccccggctcgagcagtcctgcctcagcctccggagtagctgggaccacaggttcatgccaccatggccagccaacttttgcatgttttgtagagatggggtctcacagtgttgcccaggctggtctcaaactcctgggctcaggcgatccacctgtctcagcctcccagagtgctgggattacaattgtgagccaccacgtccagctggaagggtcaacatcttttacattctgcaagcacatctgcattttcaccccacccttcccctccttctccctttttatatcccatttttatatcgatctcttattttacaataaaactttgctgccacctgtgtgtctgaggggtg

SEQ ID NO: 12 - protein, 393 aa, Homo sapiens [sequence omitted]

SEQ ID NO: 13 - protein, 393 aa, Homo sapiens [sequence omitted]

SEQ ID NO: 14 - DNA, 6053 bp, Homo sapiens
gggggaggagccaagatggccgaataggaacagctccggtctacagctcccagcgtgagcgacgcagaagacggtgatttctgcatttccatctgaggtaccgggttcatctcactagggagtgccagacagtgggcgcaggccagtgtgtgtgcgcaccgtgcgcgagccgaagcagggcgaggcattgcctcacctgggaagcgcaaggggtcagggagttccctttctgagtcaaagaaaggggtgacggtcgcacctggaaaatcgggtcactcccacccgaatattgcgcttttcagaccggcttaagaaacggcgcaccacgagactatatcccacacctggctcggagggtcctacgcccacggaatctcgctgattgctagcacagcagtctgagatcaaactgcaaggcggcaacgaggctgggggaggggcgcccgccattgcccaggcttgcttaggtaaacaaagcagccgggaagctcgaactgggtggagcccaccacagctcaaggaggcctgcctgcctctgtaggctccacctctgggggcagggcacagacaaacaaaaaggcagcagtaacctctgcagacttaagtgtccctgtctgacagctttgaagagagcagtggttctcccagcacgcagctggagatctgagaacgggcagacagactgcctcctcaagtgggtccctgactcctgacccccgagcagcctaactgggaggcaccccccagcaggggcacactgacacctcacacggcagggtattccaacagacctgcagctgagggtcctgtctgttagaaggaaaactaacaaccagaaaggacatctacaccgaaaacccatctgtacatcaccatcatcaaagaccaaaagtagataaaaccacaaagatggggaaaaaacagaacagaaaaactggaaactctaaaacgcagagcgcctctcctcctccaaaggaacgcagttcctcaccagcaacagaacaaagctggatggagaatgattttgacgagctgagagaagaaggcttcagacgatcaaattactctgagctacgggaggacattcaaaccaaaggcaaagaagttgaaaactttgaaaaaaatttagaagaatgtataactagaataaccaatacagagaagtgcttaaaggagctgatggagctgaaaaccaaggctcgagaactacgtgaagaatgcagaagcctcaggagccgatgcgatcaactggaagaaagggtatcagcaatggaagatgaaatgaatgaaatgaagcgagaagggaagtttagagaaaaaagaataaaaagaaatgagcaaagcctccaagaaatatgggactatgtgaaaagaccaaatctacgtctgattggtgtacctgaaagtgatgtggagaatggaaccaagttggaaaacactctgcaggatattatccaggagaacttccccaatctagcaaggcaggccaacgttcagattcaggaaatacagagaacgccacaaagatactcctcgagaagagcaactccaagacacataattgtcagattcaccaaagttgaaatgaaggaaaaaatgttaagggcagccagagagaaaggtcgggttaccctcaaaggaaagcccatcagactaacagtggatctctcggcagaaaccctacaagccagaagagagtgggggccaatattcaacattcttaaagaaaagaattttcaacccagaatttcatatccagccaaactaagcttcataagtgaaggagaaataaaatactttatagacaagcaaatgttgagagattttgtcaccaccaggcctgccctaaaagagctcctgaaggaagcgctaaacatggaaaggaacaaccggtaccagccgctgcaaaatcatgccaaaatgtaaagaccattgagactaggaagaaactgcatcaactaatgagcaaaatcaccagctaacatcataatgacaggatcaaattcacacataacaatattaactttaaatataaatggactaaattctgcaattaaaagacacagactggcaagttggataaagagtcaagacccatcagtgtgctgtattcaggaaacccatctcacgtgcagagacacacataggctcaaaataaaaggatggaggaagatctaccaagccaatggaaaacaaaaaaaggcaggggttgcaatcctagtctctgataaaacagactttaaaccaacaaagatcaaaagagacaaagaaggccattacataatggtaaagggatcaattcaacaagaggagctaactatcctaaatatttatgcacccaatacaggagcacccagattcataaagcaagtcctgagtgacctacaaagagacttagactcccacacattaataatgggagactttaacaccccactgtcaacattagacagatcaacgagacagaaagtcaacaaggatacccaggaattgaactcagctctgcaccaagcagacctaatagacatctacagaactctccaccccaaatcaacagaatatacctttttttcagcaccacaccacacctattccaaaattgaccacatagttggaagtaaagctctcctcagcaaatgtaaaagaacagaaattataacaaactatctctcagaccacagtgcaatcaaactagaactcaggattaagaatctcactcaaagccgctcaactacatggaaactgaacaacctgctcctgaatgactactgggtacataacgaaatgaaggcagaaataaagatgttctttgaaaccaacgagaacaaagacaccacataccagaatctctgggacgcattcaaagcagtgtgtagagggaaatttatagcactaaatgcctacaagagaaagcaggaaagatccaaaattgacaccctaacatcacaattaaaagaactagaaaagcaagagcaaacacattcaaaagctagcagaaggcaagaaataactaaaatcagagcagaactgaaggaaatagagacacaaaaaacccttcaaaaaatcaatgaatccaggagctggttttttgaaaggatcaacaaaattgatagaccgctagcaagactaataaagaaaaaaagagagaagaatcaaatagacacaataaaaaatgataaaggggatatcaccaccgatcccacagaaatacaaactaccatcagagaatactacaaacacctctacgcaaataaactagaaaatctagaagaaatggatacattcctcgacacatacactctcccaagactaaaccaggaagaagttgaatctctgaatagaccaataacaggctctgaaattgtggcaataatcaatagtttaccaaccaaaaagagtccaggaccagatggattcacagccgaattctaccagaggtacatggaggaactggtaccattccttctgaaactattccaatcaatagaaaaagagggaatcctccctaactcattttatgaggccagcatcattctgataccaaagccgggcagagacacaaccaaaaaagagaattttagaccaatatccttgatgaacattgatgcaaaaatcctcaataaaatactggcaaaccgaatccattagcacatcaaaaagcttatccaccatgatcaagtgggcttcatccctgggatgcaaggctggttcaatatacgcaaatcaataaatgtaatccagcatataaacagagccaaagacaaaaaccacatgattatctcaatagatgcagaaaaagcctttgacaaaattcaacaacccttcatgctaaaaactctcaataaattaggtattgatgggacgtatttcaaaataataagagctatctatgacaaacccacagccaatatcatactgaatgggcaaaaactggaagcattccctttgaaaaccggcacaagacagggatgccctctctcaccgctcctattcaacatagtgttggaagttctggccagggcaatcaggcaggagaaggaaataaagggtattcaattaggaaaagaggaagtcaaattgtccctgtttgcagacgacatgattgtatatctagaaaaccccatcgtctcagcccaaaatctccttaagctgataagcaacttcagcaaagtctcaggatacaaaatcaatgtacaaaaatcacaagcattcttatacaccaacaacagacaaacagagagccaaatcatgggtgaactcccattcgtaattgcttcaaagagaataaaatacctaggaatccaacttacaagggatgtgaaggacctcttcaaggagaactacaaaccactgctcaaggaaataaaagaggacacaaacaaatggaagaacattccatgctcatgggtaggaagaatcaatatcgtgaaaatggccatactgcccaaggtaatttacagattcaatgccatccccatcaagctaccaatgactttcttcacagaattggaaaaaactactttaaagttcatatggaaccaaaaaagagcccgcattgccaagtcaatcctaagccaaaagaacaaagctggaggcatcacactacctgacttcaaactatactacaaggctacagtaaccaaaacagcatggtactggtaccaaaacagagatatagatcaatggaacagaacagagccctcagaaataatgccgcatatctacaactatctgatctttgacaaacctgagaaaaacaagcaatggggaaaggattccctatttaataaatggtgctgggaaaactggctagccatatgtagaaagctgaaactggatcccttccttacaccttatacaaaaatcaattcaagatggattaaagatttaaacgttaaacctaaaaccataaaaaccctagaagaaaacctaggcattaccattcaggacataggcgtgggcaaggacttcatgtccaaaacaccaaaagcaatggcaacaaaagacaaaattgacaaatgggatctaattaaactaaagagcttctgcacagcaaaagaaactaccatcagagtgaacaggcaacctacaacatgggagaaaattttcgcaacctactcatctgacaaagggctaatatccagaatctacaatgaactcaaacaaatttacaagaaaaaaacaaacaaccccatcaaaaagtgggtgaaggacatgaacagacacttctcaaaagaagacatttatgcagccaaaaaacacatgaagaaatgctcatcatcactggccatcagagaaatgcaaatcaaaaccactatgagatatcatctcacaccagttagaatggcaatcattaaaaagtcaggaaacaacaggtgctggagaggatgcggagaaataggaacacttttacactgttggtgggactgtaaactagttcaaccattgtggaagtcagtgtggcgattcctcagggatctagaactagaaataccatttgacccagccatcccattactgggtatatacccaaatgagtataaatcatgctgctataaagacacatgcacacgtatgtttattgcggcactattcacaatagcaaagacttggaaccaacccaaatgtccaacaatgatagactggattaagaaaatgtggcacatatacaccatggaatactatgcagccataaaaaatgatgagttcatatcctttgtagggacatggatgaaattggaaaccatcattctcagtaaactatcgcaagaacaaaaaaccaaacaccgcatattctcactcataggtgggaattgaacaatgagatcacatggacacaggaaggggaatatcacactctggggactgtggtggggtcgggggaggggggagggatagcattgggagatatacctaatgctagatgacacattagtgggtgcagcgcaccagcatggcacatgtatacatatgtaactaacctgcacaatgtgcacatgtaccctaaaacttagagtataataaaaaaaaaaaaaaaaaaaaaaaaaaaaaaa

SEQ ID NO: 15 - DNA, 20 bp, Artificial Sequence
  Forward primer for LINE 1 gene
aaagccgctcaactacatgg

SEQ ID NO: 16 - DNA, 21 bp, Artificial Sequence
  Reverse primer for LINE 1 gene
tgctttgaatgcgtcccagag

SEQ ID NO: 17 - DNA, 20 bp, Artificial Sequence
  Forward primer for CYP2D6 gene
gcttctccgtctccaccttg

SEQ ID NO: 18 - DNA, 18 bp, Artificial Sequence
  Reverse primer for CYP2D6 gene
tgcccttctgcccatcac

SEQ ID NO: 19 - DNA, 20 bp, Artificial Sequence
  Forward primer for GSTT1 gene
catcctgctctacctgacgc

SEQ ID NO: 20 - DNA, 19 bp, Artificial Sequence
  Reverse primer for GSTT1 gene
gcttctccgcagagtcgtg

SEQ ID NO: 21 - DNA, 17 bp, Artificial Sequence
  Forward primer for GSTM1 gene
atcacccagagcaacgc

SEQ ID NO: 22 - DNA, 16 bp, Artificial Sequence
  Reverse primer for GSTM1 gene
acagcccagccaagga

SEQ ID NO: 23 - DNA, 20 bp, Artificial Sequence
  Forward primer for UGT2B17 gene
gaattcatcatgatcaaccg

SEQ ID NO: 24 - DNA, 20 bp, Artificial Sequence
  Reverse primer for UGT2B17 gene
acaggcaacattttgtgatc

SEQ ID NO: 25 - DNA, 17 bp, Artificial Sequence
  Forward primer for SULTA1 gene
taatccgagcctccact

SEQ ID NO: 26 - DNA, 18 bp, Artificial Sequence
  Reverse primer for SULTA1 gene
gaagtccacggtctcctc

SEQ ID NO: 27 - DNA, 23 bp, Artificial Sequence
  Forward primer for CYP2D6 gene
tcctccttccacctgctcactcc

SEQ ID NO: 28 - DNA, 23 bp, Artificial Sequence
  Reverse primer for CYP2D6 gene
ccctgacactccttcttgcctcc

SEQ ID NO: 29 - DNA, 23 bp, Artificial Sequence
  Forward primer for GSTT1 gene
agaccgaacagagaaaggtgagc

SEQ ID NO: 30 - DNA, 23 bp, Artificial Sequence
  Reverse primer for GSTT1 gene
gaactggaatagcaggaaggcaa

SEQ ID NO: 31 - DNA, 22 bp, Artificial Sequence
  Forward primer for GSTM1 gene
cctcaaccacatgtcccctctc

SEQ ID NO: 32 - DNA, 22 bp, Artificial Sequence
  Reverse primer for GSTM1 gene
tctgtcctcactgacctcccaa

SEQ ID NO: 33 - DNA, 22 bp, Artificial Sequence
  Forward primer for UGT2B17 gene
tttctgttccctccttcctatg

SEQ ID NO: 34 - DNA, 22 bp, Artificial Sequence
  Reverse primer for UGT2B17 gene
caagtccttttttttgacctcc

SEQ ID NO: 35 - DNA, 24 bp, Artificial Sequence
  Forward primer for SULTA1 gene
aaggtcacagcatcctgagaacat

SEQ ID NO: 36 - DNA, 24 bp, Artificial Sequence
  Reverse primer for SULTA1 gene
gccagaaatactatcatccccatc

SEQ ID NO: 37 - DNA, 19 bp, Artificial Sequence
  Forward primer for CYP2D6 gene
gccttcgccaaccactccg

SEQ ID NO: 38 - DNA, 20 bp, Artificial Sequence
  Reverse primer for CYP2D6 gene
aaatcctgctcttccgaggc

SEQ ID NO: 39 - DNA, 24 bp, Artificial Sequence
  Forward primer for SULTA1 gene
gttggctctgcagggtttctagga

SEQ ID NO: 40 - DNA, 26 bp, Artificial Sequence
  Reverse primer for SULTA1 gene
cccaaaccccctgctggccagcaccc

SEQ ID NO: 41 - DNA, 20 bp, Artificial Sequence
  Forward primer for TP53 gene
atctacagtcccccttgccg

SEQ ID NO: 42 - DNA, 20 bp, Artificial Sequence
  Reverse primer for TP53 gene
gcaactgaccgtgcaagtca

(19) United States
(12) Patent Application Publication
(10) Pub. No.: US 2021/0139993 A1
(43) Pub. Date: May 13, 2021

(54) METHOD FOR PREDICTING SURVIVAL IN CHILDREN WITH ACUTE LYMPHOBLASTIC LEUKEMIA

(71) Applicant: UNIVERSITAT AUTONOMA DE BARCELONA, Bellaterra (Cerdanyola del Valles) (ES)

(72) Inventors: Maria Rosa CABALLÍN FERNÁNDEZ, Bellaterra (Cerdanyola del Valles) (ES); Gemma ARMENGOL ROSELL, Barcelona (ES)

(73) Assignee: UNIVERSITAT AUTONOMA DE BARCELONA, Bellaterra (Cerdanyola del Valles) (ES)

(21) Appl. No.: 16/629,919

(22) PCT Filed: Jun. 21, 2018
(86) PCT No.: PCT/EP2018/066517

§ 371 Date: Jan. 9, 2020

(30) Foreign Application Priority Data
Jul. 11, 2017 (EP) 17382455.8

(51) Int. Cl. C12Q 1/6886

(52) U.S. Cl. CPC C12Q 1/6886; C12Q 2600/106; C12Q 2600/156

(57) ABSTRACT

The invention relates to methods for predicting the clinical outcome of cancer patients, and in particular of acute lymphoblastic leukaemia (ALL) patients, in response to a therapy against ALL, preferably conventional chemotherapy, more preferably based on glucocorticoids, said methods based on the presence of particular polymorphism in genes coding for drug-metabolizing enzymes and apoptotic proteins. The invention relates as well to method for predicting the efficacy of a therapy based on conventional glucocorticoids as well as to method for personalized medicine in patients carrying said polymorphisms.

The invention belongs to the field of methods for predicting the clinical outcome of a patient suffering from acute lymphoblastic leukaemia (ALL) in response to a conventional therapy. The invention also relates to determining polymorphisms in genes encoding drug-metabolizing enzymes and apoptotic proteins able for predicting the response to standard chemotherapeutic treatment of ALL, preferably in children suffering ALL.

### BACKGROUND ART

Acute lymphoblastic leukemia (ALL) is the most common cancer in children, representing 32% of all childhood malignances. Progressive intensification of multiple drug chemotherapeutic regimens has improved outcomes for children with ALL; however, up to 20% of patients relapse. It is known that children with ALL respond differently to chemotherapy. Synthetic glucocorticoids, such as dexamethasone and prednisone, are the keystone in the treatment of ALL in children. Response to these agents is probably the most important prognostic factor for outcome in this disease. Previous studies have observed that outcome of children with ALL might be associated with polymorphisms in genes involved in drug metabolism [e.g. P450 cytochrome (CYP), glutathione S-methyl transferase (GST), sulfotransferase (SULT), and UDP glucuronyl transferase (UGT)], DNA repair or immune surveillance processes, among others. However, these results are still controversial, since they lack confirmation, except for thiopurine S-methyltransferase (TPMT), which is routinely determined before 6-mercaptopurine administration. Nevertheless, despite extensive analysis, the underlying allelic variants responsible for ALL outcome, remain elusive.

Most of the studies performed so far have analyzed only single nucleotide polymorphisms (SNPs), except for GST and UGT genes, because the analysis was more straightforward. Among all the polymorphisms that have been reported to be possibly associated with treatment outcome in ALL, some are SNPs causing an increase or decrease in gene expression, which would have the same expected effect in the cell as an increase or decrease in the number of copies of the gene (CNV). Moreover, polymorphic copy number variants (CNVs) are very plausible candidate genetic variants useful to be considered. Of the genes with such SNPs, there are two that present inter-individual CNVs according to the Database of Genomic Variants (http://dgv.tcag.ca/dgv/app/): CYP2D6, which can present up to twelve copies and SULT1A1, up to five copies. Moreover, most studies investigating the effect of genetic polymorphisms in GSTT1, GSTM1, and UGT2B17 genes on ALL outcome distinguished between null and non-null genotypes, but the effect of the exact gene copy number is never considered (Nørskov M S, et al. Clin. Biochem. 2009; 42:201-209; Gaedigk A, et al. Pharmacogenomics. 2012; 13:91-111). It is known that the proteins coded by these genes contribute to the metabolism and disposition of many drugs used to treat childhood ALL (Pharmacogenomics Knowledge Base, www.pharmgkb.org). In this sense, although GSTM1 is mainly known as a detoxification enzyme, it has also been described as a downregulator of pro-apoptotic pathways.

Hosono et al., (Hosono N, et al. Cancer Sci. 2010; 101:767-773) reported that GSTM1-expressing ALL cell lines exhibited decreased sensitivity to some antileukemic drugs, through inhibition of apoptosis instead of through glutathione transfer. The key mediator of apoptosis is p53, which modulates DNA repair and apoptosis upon DNA damage. It is known that the GST family of enzymes catalyzes the inactivation of several antileukemic agents through conjugation with glutathione. For example, the parent drug or metabolites of vincristine, etoposide, cyclophosphamide, anthracyclines (such as daunorubicin) and glucocorticosteroids, widely used in childhood ALL therapy, are substrates for GSTs37. Likely, GSTM1 non-null genotype would lead to increased inactivation of these drugs, compared to null genotype. This means lower drug exposure and subsequent poorer treatment efficacy and poorer survival. So far, GSTM1 null genotype has been associated with improved childhood ALL outcome in some studies (Stanulla M, et al. Blood. 2000; 95(4):1222-1228; Rocha J C C. et al. Pharmacogenetics. 2005; 105(12):4752-4758; Franca R, et al. Pharmacogenomics. 2012; 13(16):1905-16), but not in others (Davies S M, et al. Blood. 2002; 100(1):67-71; Anderer G, et al. Pharmacogenetics. 2000; 10(8):715-26; Krajinovic M, et al. Clin. Cancer Res. 2002; 8(3):802-810). In this sense, there is no explanation for these discrepancies, although one explication could be the different multidrug therapeutic strategy used in the patients studied.

It is well known that a common polymorphism of TP53 in codon 72 produces an arginine (Arg or R) to proline (Pro or P) change that diminishes p53 apoptotic activity and this polymorphism has been reported to be associated with hematological (Ørsted D D, et al. J. Exp. Med. 2007; 204(6):1295-1301; Do T N, et al. Cancer Genet. Cytogenet. 2009; 195(1):31-36; Tian X, et al. Sci. Rep. 2016; 6(24097):1-6) and non-hematological cancer risk (Khan M H, et al. Genet. Res. (Camb). 2015; 97) and with response to chemotherapy and survival in several types of tumors (Rodrigues P, et al. Mol. Cell. Biochem. 2013; 379(1-2):181-190; Liu L, et al. J. Thorac. Oncol. 2011; 6(11):1793-800; Toffoli G, et al. Clin. Cancer Res. 2009; 15(10):3550-3556; Cescon D W, et al. Clin. Cancer Res. 2009; 15(9):3103-3109), even though the impact of this polymorphism seems to depend on the type of cancer and on the particular treatment.

Identification of children with acute lymphoblastic leukemia prone to have poor response to chemotherapy is important to improve individualization of therapy. Despite many pharmacogenetic studies carried out in recent years, clinically useful genetic predictors of ALL treatment response need to be identified. Therefore, there is a need in the art for identifying markers for predicting the clinical outcome of a patient, preferably children suffering from ALL in response to a therapy which solves the problems of the methods known to date.

### SUMMARY OF THE INVENTION

The inventors have identified that polymorphisms, preferably CNVs and SNP in genes encoding drug-metabolizing enzymes and apoptotic proteins, such as GSTM1 and TP53 genes, are significantly associated with outcome of patients, preferably children, suffering ALL in response to a therapy against ALL, preferably wherein the therapy is a conventional or standard chemotherapeutic treatment.

In the present invention, the inventors screened 173 Caucasian children with ALL for the presence of polymorphisms on gene copy number and/or SNPs in the genes CYP2D6 (SEQ ID NO: 1), GSTT1 (SEQ ID NO: 3), GSTM1 (SEQ ID NO: 5), UGT2B17 (SEQ ID NO: 7), SULT1A1 (SEQ ID NO: 9) and TP53 (SEQ ID NO: 11), and studied the association of the genotypes with the clinical outcome such as, early-treatment response, end-of-induction minimal residual disease (MRD), relapse, and overall survival. The inventors found that genetic variants in GSMT1 (SEQ ID NO: 5) and TP53 (SEQ ID NO: 11) genes are associated with survival in childhood ALL. Particularly, they found that the GSTM1 (SEQ ID NO: 5) non-null genotype and the p53Pro variant at codon 72 at the polymorphism rs1042522 in TP53 (enough in heterozygosis, Pro/Arg or Pro/Pro genotype) (SEQ ID NO: 13) are, in combination, useful as genetic predictors of poor or bad clinical outcome in the ALL treatment according to Kaplan-Meier analysis. This can help the physicians in the design of individualized therapy of these patients.

In addition, the inventors evaluated the in vitro effect of the polymorphisms GSTM1 (SEQ ID NO: 5) CNV and the TP53 Arg72Pro SNP (SEQ ID NO: 13) on the response to an anticancer agent, such as the glucocorticoid dexamethasone, in a leukemic cell line, confirming the results observed in patients.

Thus, in a first aspect, the invention relates to a method for predicting the clinical outcome of a patient suffering from ALL, preferably in response to a to a therapy against ALL, more preferably in response to a conventional chemotherapy, preferably wherein the conventional chemotherapy comprises at least a glucocorticoid compound, which comprises determining in a biological sample from said patient the polymorphisms in GSTM1 and TP53 genes, preferably wherein the polymorphisms are selected from the GSTM1 (SEQ ID NO: 5) CNV and the TP53 Arg72Pro SNP (SEQ ID NO: 13) wherein
the presence of at least one copy of the GSTM1 gene (also named as GSTM1 non-null genotype) (SEQ ID NO: 5) and the presence of p53Pro variant at codon 72 at the polymorphism rs1042522 in TP53 (enough in heterozygosis) Pro/Arg or Pro/Pro genotype of TP53 Arg72Pro polymorphism (SEQ ID NO: 13) is indicative of unfavourable clinical outcome, or
the absence of GSTM1 (SEQ ID NO: 5) gene and the presence of Arg/Arg genotype of TP53 Arg72Pro polymorphism (SEQ ID NO: 12) is indicative of favourable clinical outcome.

In a preferred embodiment of the first aspect of the invention, the therapy against ALL is preferably a conventional chemotherapy, more preferably a conventional chemotherapy comprises at least a glucocorticoid compound and more preferably a combination of glucocorticoids.

In a second aspect, the invention relates to a method for predicting the efficacy of a therapy against ALL, preferably wherein the therapy is a conventional chemotherapy, more preferably based on a glucocorticoid compound, for treating ALL which comprises determining in a biological sample from said patient the polymorphisms selected from the copy number of GSTM1 (SEQ ID NO: 5) gene and Arg72Pro TP53 (SEQ ID NO: 13) wherein
the presence of at least one copy of the GSTM1 gene (SEQ ID NO: 5) and the presence of Pro/Arg or Pro/Pro (SEQ ID NO: 13) genotype is indicative of poor efficacy of the therapy, or
the absence of GSTM1 gene (also named as GSTM1 null genotype) and the presence of Arg/Arg (SEQ ID NO: 12) genotype is indicative of a good efficacy of the therapy.

In a third aspect, the invention relates to a method for selecting a patient suffering from ALL for a therapy non-based on a conventional chemotherapy agent, preferably non-based on a glucocorticoid compound, comprising determining in a biological sample from said patient the presence of at least one copy of the GSTM1 (SEQ ID NO: 5) gene and the presence of Arg72Pro TP53 (SEQ ID NO: 13) genotype. Alternatively, in another aspect, the invention relates to a method for selecting a patient suffering from ALL for a therapy based on a conventional chemotherapy agent, preferably based on a glucocorticoid compound, comprising determining in a biological sample from said patient the absence of the GSTM1 (SEQ ID NO: 5) gene and the presence of Arg/Arg genotype (SEQ ID NO: 12).

In a fourth aspect, the invention relates to a therapy non-based on a conventional chemotherapy, preferably on a non-glucocorticoid compound, for use in the treatment of a patient suffering from ALL, wherein said patient has been selected by the third method of the present invention. Alternatively, the invention relates to a therapy based on a conventional chemotherapy, preferably on a glucocorticoid compound, for use in the treatment of a patient suffering from ALL, wherein said patient has been selected by the third method of the present invention.

In another aspect, the invention relates to a kit for in vitro predicting the clinical outcome of a patient suffering from ALL, preferably in response to a therapy against ALL, preferably chemotherapy, and more preferably in response to a glucocorticoid compound, for in vitro predicting the efficacy of a conventional chemotherapy in a patient suffering from ALL, or for in vitro selecting a patient suffering from ALL for a conventional chemotherapy said kit comprising reagents for analysing a biological sample from said subject for the copy number of the GSTM1 gene and for the genotype of the Arg72Pro polymorphism in the TP53 gene. In another aspect the present invention also relates to the use in vitro of the kit of the invention for predicting the clinical outcome of a patient suffering from ALL, preferably in response to a therapy against ALL, preferably chemotherapy, and more preferably in response to a glucocorticoid compound, for in vitro predicting the efficacy of a conventional chemotherapy in a patient suffering from ALL, or for in vitro selecting a patient suffering from ALL for a conventional chemotherapy said kit comprising reagents for analysing a biological sample from said subject for the copy number of the GSTM1 gene and for the genotype of the Arg72Pro polymorphism in the TP53 gene.

In another additional aspect the invention relates to the use in vitro of the polymorphisms selected from the copy number of GSTM1 gene comprising the SEQ ID NO: 5 and the rs1042522 comprising the SEQ ID NO: 13, for predicting the clinical outcome of a patient suffering from ALL, for predicting the efficacy of a conventional chemotherapy, or for selecting a patient suffering from ALL for a conventional chemotherapy.

### DESCRIPTION OF THE DRAWINGS

FIG. 1. Kaplan-Meier survival curves of childhood ALL patients according to age. Patients with less than 10 years had better survival than the others (A, OS p=0.002; B, EFS p=0.080). 1=<10 years; 2=≥10 years.

FIG. 2. Kaplan-Meier survival curves of childhood ALL patients according to cytogenetic risk group. Patients with good cytogenetic risk group had better survival than patients with intermediate risk group. Surprisingly, patients with adverse cytogenetic risk group had better survival than the others. This can be explained because five of the six cases with adverse cytogenetics harbored the t(9; 22) and imatinib treatment was successful (A, OS p=0.004; B, EFS p=0.085). 1=Adverse cytogenetic risk group; 2=Good cytogenetic risk group; 3=Intermediate cytogenetic risk group.

FIG. 3. Kaplan-Meier survival curves of childhood ALL patients according to MRD. Patients with no MRD had better survival than the others (A, OS p=0.024; B, EFS p=0.028). 1=No MRD; 2=MRD.

FIG. 4. Kaplan-Meier survival curves of childhood ALL patients according to leukemia type. Patients with B-type leukemia had better survival than the others (A, OS p=0.168). 1=B-Type; 2=T-Type.

FIG. 5. Kaplan-Meier survival curves of childhood ALL patients according to early response. Patients with good early response had better survival than the others (A, OS p=0.085; B, EFS p=0.088). 1=Good response; 2=Bad response.

FIG. 6. Kaplan-Meier survival curves of childhood ALL patients according to the GSTM1 genotype. Patients with GSTM1 null genotype had better survival than the other genotypes, both when analyzing genotypes separately (A, OS p=0.013; B, EFS p=0.047) [1=Null genotype; 2=1 copy; 3=2 copies; 4≥3 copies], or when analyzing null vs. non-null (C, OS p=0.002; D, EFS p=0.005) [5=Null genotype; 6=Non-null genotype].

FIG. 7. Kaplan-Meier survival curves of childhood ALL patients according to the TP53 genotype at codon 72. Patients with the Arg/Arg genotype had better survival than the others, even though differences were not significant (A, OS p=0.208; B, EFS p=0.110). 1=Arg/Arg; 2=Pro/Pro-Pro/Arg.

FIG. 8. Kaplan-Meier survival curves of childhood ALL patients according to the GSTM1 and TP53 genotypes. Patients with ≥1 copies of GSTM1 and TP53 Pro/Pro or Pro/Arg genotype had worse survival than the others (A, OS p=0.0005; B, EFS p=0.003). 1=Others; 2=GSTM1 and TP53 Pro/Pro-Pro/Arg.

FIG. 9. Survival analysis of Jurkat cells with the TP53 Arg or Pro variants and GSTM1 null/non-null treated with varying concentrations of dexamethasone by MTT assay. Cells with p53Pro and GSTM1 non-null showed significantly less sensitivity than cells with p53Arg and GSTM1 null at all concentrations. Bars correspond to standard deviations from six separate measurements. This experiment is representative of two independent experiments.

### DETAILED DESCRIPTION OF THE INVENTION

The authors of the present invention have found that the association between the CNV in GSTM1 gene and the presence of SNP in TP53 gene can predict the clinical outcome in children with ALL. Particularly, they found that the patients having GSTM1 non-null genotype and the polymorphism rs1042522 in combination are useful as genetic predictors of poor clinical outcome in the ALL treatment, and helping the physicians in the design of individualized therapy of these patients. This finding opens the door to new genetic predictors of clinical outcome in the treatment of children with ALL, helping the physician in the design of individualized therapy in childhood ALL. Based on these findings, the inventors have developed the methods of the present invention which will be described now in detail.

Method for Predicting the Clinical Outcome of a Patient Suffering from ALL.

In a first aspect, the invention relates to a method for predicting the clinical outcome of a patient, preferably, wherein the patient is a child, suffering from ALL, preferably in response to a therapy against ALL, more preferably in response to a conventional chemotherapy, and more preferably in response to a therapy based on glucocorticoids compounds (hereinafter, first method of the invention), which comprises determining in a biological sample from said patient the polymorphisms selected from the copy number of GSTM1 (SEQ ID NO: 5) gene and Arg72Pro polymorphism (r51042522, SEQ ID NO: 13) in TP53 gene wherein,
the presence of at least one copy of the GSTM1 gene and the presence of Pro/Arg or Pro/Pro genotype in the rs1042522 is indicative of unfavourable clinical outcome, or
the absence of GSTM1 gene and the presence of Arg/Arg genotype in the rs1042522 is indicative of favourable clinical outcome.

The term “prognosis” refers to the prediction of the likelihood of cancer-attributable death or progression, including recurrence, metastatic spread and drug resistance, of a disease such as ALL. The term “prediction” is used herein to refer to the likelihood that a patient will have a particular clinical outcome. As will be explained later, the clinical outcome may be positive, favourable or good (used interchangeably throughout this document) or negative, unfavourable, poor or bad (used interchangeably throughout this document). The predictive methods of the present invention can be used clinically to make treatment decisions by choosing the most appropriate treatment modalities for any particular patient. The predictive method of the present invention is a valuable tool in predicting if a patient is likely to respond favourably to a treatment regimen. As will be understood by those skilled in the art, the prediction, although preferred to be, need not be correct for 100% of the subjects to be diagnosed or evaluated. The term, however, requires that a statistically significant portion of subjects can be identified as having an increased probability of having a given outcome. Whether a subject is statistically significant can be determined without further ado by the person skilled in the art using various well known statistic evaluation tools, e.g., determination of confidence intervals, p-value determination, Student's t-test, Mann-Whitney test, etc. Preferred confidence intervals are at least 50%, at least 60%, at least 70%, at least 80%, at least 90% at least 95%. The p-values are, preferably 0.05, 0.02, 0.01 or lower.

Standard criteria (Miller, et al. Cancer, 1981; 47(1): 207-14) can be used herewith to evaluate the clinical outcome of a patient, preferably in response to a therapy. Any parameter which is widely accepted for determining the efficacy of treatments can be used for determining the clinical outcome of a patient in response to a treatment and include, without limitation:
disease-free progression which, as used herein, describes the proportion of subjects in complete remission who have had no recurrence of disease during the time period under study.
disease-free survival (DFS), as used herewith, is understood as the length of time after treatment for a disease during which a subject survives with no sign of the disease.
objective response which, as used in the present invention, describes the proportion of treated subjects in whom a complete or partial response is observed.
progression free survival which, as used herein, is defined as the time from start of treatment to the first measurement of cancer growth.
time to progression (TTP), as used herein, relates to the time after a disease is treated until the disease starts to get worse. The term “progression” has been previously defined.
Minimal residual disease (MRD), as used herein relates to a situation or condition where there is no evidence of disease in a subject, but where the subject in fact has residual tumor cells. Typically, MRD occurs after the use of chemotherapy, surgery and/or radiation therapy.
Relapse or recurrence, as used herein means that a cancer had been successfully treated has now returned. The cancer may have returned in its original location, or it may be in a new location, and
Overall survival (OS) or survival which, as used herein, is defined as the percentage of patients who survive after diagnosis of ALL

The term “patient” as used herein refers to a subject suffering from cancer, preferably for ALL. The term “subject”, as used herein, refers to all animals classified as mammals and includes, but is not restricted to, domestic and farm animals, primates and humans, e.g., human beings, non-human primates, cows, horses, pigs, sheep, goats, dogs, cats or rodents. Preferably, the subject is a male or female human of any race. The term “pediatric ALL” or “pediatric ALL patient” as referred to herein denotes children aged from one month to 18 years. The indicated age is to be understood as the age of the children at diagnosis of the ALL disease. The term “child” as used herein refers to a human person having not more than 18 years of age, and includes children from about 12 months to about 18 years of age.

The above definitions apply mutatis mutandis to the term “pediatric acute lymphoblastic leukemia (ALL)”, “childhood ALL”, or the like. For example, pediatric or childhood ALL is to be understood as ALL diagnosed in a pediatric patient aged between 1 month (including 1 month) and 18 years (including 18 years).

As previously explained, the first method of the invention allows the skilled person to predict the clinical outcome of a patient suffering ALL, preferably in response to a therapy against ALL, preferably in response to a conventional chemotherapy, and more preferably in response to a conventional chemotherapy based on glucocorticoid compounds. The term “therapy”, as used herein, refers to a therapeutic treatment, as well as a prophylactic or prevention method, wherein the goal is to prevent or reduce an unwanted physiological change or disease, such as ALL. Beneficial or desired clinical results include, but not limiting, release of symptoms, reduction of the length of the disease, stabilized pathological state (specifically not deteriorated), retard in the disease's progression, improve of the pathological state and remission (both partial and total), both detectable and not detectable. The terms “treat” and “treatment” are synonyms of the term “therapy” and can be used without distinction along the present description. “Treatment” can mean also prolong survival, compared to the expected survival if the treatment is not applied.

In a preferred embodiment, the term “standard therapy” or “conventional therapy” as used herein refers to pediatric ALL therapy using chemotherapy and/or hematopoietic stem cell transplantation (HSCT). Pediatric acute lymphoblastic leukemia (ALL), including pediatric B-precursor acute lymphoblastic leukemia and other types of pediatric B (cell) lineage ALL, and treatments thereof are reviewed e.g. in Pui C H, Clin Adv Hematol Oncol. 2006, 4: 884-6; Pui C H, Evans W E, N Engl J Med 2006, 354: 166-178; Pui C H et al., Lancet 2008, 371: 1030-1043; Pui C H, Jeha S, Nat Rev Drug Discov 2007, 6: 149-165; Henze G, von Stackelberg A, Relapsed acute lymphoblastic leukemia. In: Childhood Leukemias, C-H Pui ed. Cambridge: Cambridge University Press; 2006, p. 473-486. Further information with respect to pediatric ALL can also be found e.g. under http://www.cancer.gov or http://www.leukemia-lymphoma.org

The term “chemotherapy” as used herein denotes chemotherapy used for the treatment of acute lymphoblastic leukemia (ALL). Chemotherapy is the initial treatment of choice for ALL. Most ALL patients end up receiving a combination of different treatments. In general, cytotoxic chemotherapy for ALL combines multiple anti-leukemic drugs in various combinations. For example, the preferred anti-leukemic drugs are selected from the list consisting of: glucocorticoids such as prednisona (prednisolone), hydrocortisone and dexamethasone, vincristine, daunorubicin, L-asparaginase, methotrexate, cytarabine, mercaptopurine, cyclophosphamide, doxorubicin, tioguanine, vindesine, ifosfamide, etoposide, thioguanine, mercaptopurine or any combinations thereof. In a particular embodiment of the first method of the invention, the treatment comprises at least a glucocorticoid compound selected from the list consisting of prednisona (prednisolone), hydrocortisone, dexamethasone or any combinations thereof.

The first method of the invention comprises determining in a biological sample from said patient the polymorphisms GSTM1 CNV (SEQ ID NO: 5) and the TP53 Arg72Pro SNP (SEQ ID NO: 13).

The terms “biological sample” and “sample”, used interchangeably in the present invention, relate to any sample which can be obtained from the subject. The present method can be applied to any kind of biological sample from a subject, such as a biopsy sample, tissue, cell or biofluid such as blood, serum, plasma, saliva, semen, sputum, cerebral spinal fluid (CSF), and the like. In a particular embodiment, said sample is a biofluid sample as named as biological fluid, preferably, a peripheral blood sample and saliva, more preferably saliva. Said samples can be obtained by conventional methods well known to those of ordinary skill in the related medical arts.

As it is used in the present description, the term “polymorphism” relates to a one or more of: single nucleotide polymorphisms (SNPs); small insertions or deletions (indels); copy number variations (CNVs) and combination thereof. In a more preferred embodiment, the term polymorphism relates to single nucleotide polymorphisms and copy number variations. As used herein, “Copy Number Variation” or “CNV” refer to a DNA segment of one kilobase (kb) or larger that is present at a variable copy number in comparison with a reference genome. CNVs usually correspond to relatively large regions of the genome that have been deleted (fewer than the normal number) or duplicated/multiplicated (e.g., more than the normal copy number of 2) on certain chromosomes. In certain embodiments, the CNV increases the copy number of a gene. In another embodiment, the CNV reduces the copy number of a gene. In certain embodiments, the CNV is an inherited genetic defect. In another embodiment, the CNV is generated de novo in an individual. In certain embodiments, the CNV affects a single gene. In the present invention CNV preferably increases the copy number of a gene. As used herein, “Single nucleotide polymorphism” or “SNP” means a single nucleotide (A, C, T or G) position in a genomic sequence for which two or more alternative alleles are present at an appreciable frequency (e.g., at least 1%) in a population. In the present invention, the terms polymorphic site and SNPs can be used indistinctly. The SNPs are typically named using the accession number in the Single Nucleotide Polymorphism (SNP) database (dbSNP) at National Center for Biotechnology Information (NCBI) accessible at http://www.ncbi.nlm.nih.gov/projects/SNP/. Alternatively, the SNP can be named as well by indicating the position at the gene or genomic contig wherein the variation occurs and the type of nucleotide change that occurs at said position or the type of amino acid change in the polypeptide encoding by said gene.

The polymorphism rs1042522, Arg72Pro and P72R are used interchangeably throughout this document. This polymorphism is located in the TP53 gene (SEQ ID NO: 11), and corresponds to the polypeptide sequences SEQ ID NO: 12 and 13.

The term “allele” is used in the present description and relates to a polymorphism occurring in one and the same locus in one and the same population.

CNV and SNP genotyping is the measurement of copy number variations and single nucleotide polymorphisms (SNPs). The genotype of a subject is determined from a sample of the nucleic acid from the subject. The detection of the polymorphism in the method of the invention can be performed by means of multiple processes known by the person skilled in the art. Said methods can be found, for example, in Sambrook et al, 2001. “Molecular cloning: a Laboratory Manual”, 3rd ed., Cold Spring Harbor Laboratory Press, N.Y., Vol. 1-3. Systems and methods for the detection of polymorphisms associated to genes include, but are not limited to, hybridization methods and array technology, techniques based on mobility shift in amplified nucleic acid fragments, Single Stranded Conformational Polymorphism (SSCP), denaturing gradient gel electrophoresis (DGGE), Chemical mismatch cleavage (CMC), Restriction fragment polymorphisms (RFLPs), nucleic acid sequencing and the like. Generation of nucleic acids for analysis from samples generally requires nucleic acid amplification. Many amplification methods rely on an enzymatic chain reaction (such as a polymerase chain reaction). Real-time PCR (also known as Quantitative PCR, Real-time Quantitative PCR, or RTQ-PCR) is a method of simultaneous DNA quantification and amplification (Expert Rev. Mol. Diagn. 2005(2):209-19). DNA is specifically amplified by polymerase chain reaction. After each round of amplification, the DNA is quantified. Common methods of quantification include the use of fluorescent dyes that intercalate with double-strand DNA and modified DNA oligonucleotides (called probes or primers) that fluoresce after hybridising with a complementary DNA. In a preferred embodiment for the first method of the present invention the probes or primer are selected from the list disclosed in Table 2.

Once the polymorphic site rs1042522 and the GSTM1 CNV is determined, the first method of the invention further comprises determining whether the clinical outcome of a patient in response to conventional chemotherapy, preferably based on glucocorticoids compound will be favourable or unfavourable. This determination is carried out as follows:
if there is determined the presence of at least one copy of the GSTM1 gene (GSTM1 non-null genotype) and the presence of Pro/Arg or Pro/Pro genotype in the rs1042522, then this is indicative of unfavourable clinical outcome of the therapy, or
if there is determined the absence of GSTM1 gene (GSTM1 null genotype) and the presence of Arg/Arg genotype in the rs1042522, then this is indicative of favourable clinical outcome of the therapy.

The term “unfavourable clinical outcome”, “negative clinical outcome” or “poor prognosis” as used herein, refers to not obtaining beneficial or desired clinical results which can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. Additionally, these terms also mean that the therapy has a poor efficacy on said patient.

The term “favourable clinical outcome”, “positive clinical outcome” or “good prognosis” as used herein, refers to obtaining beneficial or desired clinical results which can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. Additionally, these terms also mean that the therapy has a good efficacy on said patient.

As used herein, and confined to diploids, “homozygous” is defined as a genetic condition existing when identical alleles reside at corresponding loci on homologous chromosomes.

Method for Predicting the Efficacy of a Conventional Chemotherapy Treatment in Childhood ALL.

In another aspect, the invention relates to a method for predicting the efficacy of a conventional chemotherapy treatment, preferably wherein the chemotherapy comprising at least one glucocorticoid, for treating ALL (hereinafter, second method of the invention), which comprises determining in a biological sample from said patient the polymorphisms in GSTM1 (SEQ ID NO: 5) and TP53 (SEQ ID NO: 11) genes, preferably the polymorphisms selected from the GSTM1 (SEQ ID NO: 5) CNV and the TP53 Arg72Pro SNP (SEQ ID NO: 11) wherein:
the presence of at least one copy of the GSTM1 gene and the presence of Pro/Arg or Pro/Pro genotype in the rs1042522 is indicative of a poor efficacy of the therapy, or
the absence of GSTM1 gene and the presence of Arg/Arg genotype in the rs1042522 is indicative of a good efficacy of the therapy.

The expression “predicting the efficacy of a therapy”, as used herein, refers to determine the clinical outcome of a patient suffering cancer in response to a therapy based on a conventional chemotherapy in ALL, preferably glucocorticoid-based compound.

Method for the Selection of Patients

In another aspect, the invention relates to a method for selecting a patient suffering from ALL for a therapy against ALL, more preferably for a conventional chemotherapy, and more preferably for a therapy based on glucocorticoids compounds (hereinafter, third method of the invention) comprising determining in a biological sample from said patient the polymorphisms in GSTM1 and TP53 genes, preferably wherein the polymorphisms selected from the GSTM1 CNV (GSTM1 non-null genotype) and the TP53 Arg72Pro SNP (Pro/Arg or Pro/Pro genotype).

All the particular embodiments disclosed previously for the first method of the invention are also applicable to the second and third methods of the invention, such as the conventional chemotherapy based preferably on glucocorticoids compounds, the sample from a patient is a biofluid sample, preferably, saliva; the determination of the CNV and SNP comprises the use of at least one oligonucleotide, probe or primer specific for one of the alleles of said polymorphics site or sites; etc. Likewise, the definitions and techniques provided for the first method of the invention are also applicable to the second and third methods of the invention.

Use of Reagents Suitable for Detection of the CNV and SNP for the Methods of the Present Invention.

In another aspect, the invention relates to the use of a reagent suitable for the detection of the sequence of the polymorphic site rs1042522 and the CNV for the GTSM1 gene for predicting the clinical outcome of a patient in response to a conventional chemotherapy based on a glucocorticoid compound, for predicting the efficacy of a conventional chemotherapy based on a glucocorticoid compound, or for selecting a patient suffering from ALL for a conventional chemotherapy based on a glucocorticoid compound.

The reagent suitable for the detection of the GSTM1 CNV and the TP53 Arg72Pro SNP of the present invention may be a probe, a primer or a oligonucleotide which is able to distinguish a particular form of the gene or the presence or a particular variance or variances, e.g., by differential binding or hybridization. Thus, exemplary probes include nucleic acid hybridization probes, peptide nucleic acid probes, nucleotide-containing probes which also contain at least one nucleotide analogue, and antibodies, e.g., monoclonal antibodies, and other probes as discussed herein. Those skilled in the art are familiar with the preparation of probes with particular specificities. Those skilled in the art will recognize that a variety of variables can be adjusted to optimize the discrimination between two variant forms of a gene, including changes in salt concentration, temperature, pH and addition of various compounds that affect the differential affinity of GC vs. AT base pairs (See Current Protocols in Molecular Biology by F. M. Ausubel, R. Brent, R. E. Kingston, D. D. Moore, J. G. Seidman, K Struhl and V. B. Chanda (Editors), John Wiley and Sons.). Such a nucleic acid hybridization probe may span two or more variance sites. Unless otherwise specified, a nucleic acid probe can include one or more nucleic acid analogs, labels or other substituents or moieties so long as the base-pairing function is retained. In a more preferred embodiment of the methods of the present invention, the probes are selected from the primers disclosed in Table 2. Additionally, the detection of the polymorphisms of the present invention can be performed by means of multiple processes known by the person skilled in the art and mentioned above.

Kits of the Invention

The present invention also contemplates the preparation of kits for use in accordance with the present invention. Suitable kits include various reagents for use in accordance with the present invention in suitable containers and packaging materials, including tubes, vials, and shrink-wrapped and blow-molded packages. Materials suitable for inclusion in an exemplary kit in accordance with the present invention comprise one or more of the following: gene specific PCR primer pairs (oligonucleotides) that anneal to DNA or cDNA sequence domains that flank the genetic polymorphisms of the present invention, reagents required to discriminate between the various possible alleles in the sequence domains amplified by PCR or non-PCR amplification (e.g., restriction endonucleases, oligonucleotide that anneal preferentially to one allele of the polymorphism, including those modified to contain enzymes or fluorescent chemical groups that amplify the signal from the oligonucleotide and make discrimination of alleles more robust); reagents required to physically separate products derived from the various alleles (e.g. agarose or polyacrylamide and a buffer to be used in electrophoresis, HPLC columns, SSCP gels, formamide gels or a matrix support for MALDI-TOF). Specifically contemplated are kits comprising two or more polymorphism-specific or allele-specific oligonucleotides or oligonucleotide pairs, wherein each polymorphism-specific or allele-specific oligonucleotide or oligonucleotide pair is directed to one of the polymorphisms recited herein.

For example, the present invention contemplates a kit comprising one or more polymorphism-specific or allele-specific oligonucleotide (probe or primer) or oligonucleotide pair (probes or primers) directed to one or more of the polymorphisms rs1042522 and the CNV for the GSTM1 gene. Preferably, the probes are selected from the primers disclosed in Table 2.

It will be appreciated that in this context the term “directed to” means an oligonucleotide or oligonucleotide pair capable of identifying the allele present at the polymorphism.

Unless otherwise defined, all technical and scientific terms used herein have the same meaning as commonly understood by one of ordinary skilled in the art to which this invention belongs. Methods and materials similar or equivalent to those described herein can be used in the practice of the present invention. Throughout the description and claims the word “comprise” and its variations are not intended to exclude other technical features, additives, components, or steps. Additional objects, advantages and features of the invention will become apparent to those skilled in the art upon examination of the description or may be learned by practice of the invention. The following examples, drawings and sequence listing are provided by way of illustration and are not intended to be limiting of the present invention.

### EXAMPLES

The following examples are offered to illustrate, but not to limit, the claimed invention. It is understood that the examples and embodiments described herein are for illustrative purposes only, and persons skilled in the art will recognize various reagents or parameters that can be altered without departing from the spirit of the invention or the scope of the appended claims.

### Example 1

### I. Materials and Methods

Patients

One hundred and seventy-three patients diagnosed with ALL and younger than 18 years were recruited from Sant Joan de Déu Hospital, Barcelona, Spain and from Cruces Hospital, Barakaldo, Spain between 1996 and 2012. Patients were all from Caucasian descent. All their parents were informed about the study and they provided their consent in accordance with the Declaration of Helsinki. The research was approved by the ethics committees of all participant hospitals. Samples were extracted when patients were at complete remission or without cytogenetic aberrations affecting chromosomes where the polymorphisms are located. Clinical data were collected at diagnosis, including sex, age, leukemia type (T-cell, B-cell or mixed), WBC count, cytogenetic risk group and treatment protocol (Table 1).

The different cytogenetic risk groups were classified according to the criteria from the Spanish Society of Pediatric Hematology and Oncology as follow: Presence of t(9; 22), t(4; 11) or MLL rearrangements at 11q23, or hipodiploidy (<44 chromosomes) was considered adverse group. Presence of 412; 21) or hiperdiploidy (>50 chromosomes) was considered favorable and all other cases were considered to belong to intermediate cytogenetic risk group.

Patients were risk assigned and treated according to the following protocols: SHOP-1994, SHOP-1999 or SHOP-2005 (Sociedad Espanola de Hematologia y Hemoterapia; http://www.sehh.es/es/). Treatment efficacy was assessed by means of early response, MRD, relapse and survival. Early response was based on the percentage of leukemic blasts in bone marrow on day 14 (<5% corresponds to a good response). MRD was determined in bone marrow at the end of remission induction by flow cytometry and positivity was defined as the presence of 0.01% or more ALL cells.

TABLE 1
Patient characteristics, clinical-biological
data and treatment efficacy
parameters of children with ALL included
in the present study. The difference
between the number of subjects of each
characteristic and the total number of
patients included in the study (n = 173)
corresponds to lack of information.

| | Number of subjects |
|---|---|
| | and frequency (%) |
| Sex (n = 173) | |
| Female | 77 (44.5%) |
| Male | 96 (55.5%) |
| Age (n = 173) | |
| 0-10 years | 135 (78%) |
| >10 years | 38 (22%) |
| Leukemia type (n = 162) | |
| T-cell | 20 (12.3%) |
| B-cell | 142 (87.7%) |
| WBC count (×10⁹/L) (n = 157) | |
| ≤50 | 126 (80.2%) |
| >50 | 31 (19.8%) |
| Cytogenetic risk group (n = 140) | |
| Favorable | 63 (45%) |
| Intermediate | 71 (50.7%) |
| Adverse | 6 (4.3%) |
| Treatment protocol (n = 173) | |
| SHOP-1994 | 4 (2.3%) |
| SHOP-1999 | 23 (13.3%) |
| SHOP-2005 | 146 (84.4%) |
| Early response* (n = 158) | |
| Good | 129 (81.6%) |
| Bad | 29 (18.4%) |
| MRD* (n = 110) | |
| Positive | 12 (10.9%) |
| Negative | 98 (89.1%) |
| Relapse (n = 172) | |
| Yes | 20 (11.6%) |
| No | 152 (88.4%) |
| OS (n = 173) | |
| Alive | 160 (92.5%) |
| Exitus | 13 (7.5%) |

WBC: white blood cell; MRD: minimal residual disease; OS: overall survival.

DNA Isolation

DNA was extracted either from bone marrow cell nuclei in fixative (acetic acid-methanol, 1:3) or from peripheral blood cells at remission using the QIAamp DNA Mini Kit (Qiagen, Hilden, Germany) and following manufacturer's instructions.

CNV Genotyping by Quantitative Real-Time Polymerase Chain Reaction (qPCR)

Relative quantification of CYP2D6 (SEQ ID NO: 1), GSTT1 (SEQ ID NO: 3), GSTM1 (SEQ ID NO: 5), UGT2B17 (SEQ ID NO: 7), and SULT1A1 (SEQ ID NO: 9) gene copy number was performed by qPCR. DNA concentrations were normalized referring to the constant copy number reference LINE1 (SEQ ID NO: 14). Moreover, the relative gene of interest (GOI) copy number level was also normalized to a human genomic DNA sample from a healthy donor as calibrator.

We used specific primers targeting unique sequences of 110-201 bp within the GOI and the reference sequence as given in Table 2. Specificity of each primer pair was verified using the Basic Local Alignment Search Tool (www.ncbi.nlm.nih.gov/BLAST/).

TABLE 2
Primer sequences and product size
for qPCR, long-PCR and PCR-RFLP.

| | | | | Product |
|---|---|---|---|---|
| Technique | Primer | Forward (5′→3′) | Reverse (5′→3′) | size (bp) |
| qPCR | LINE1 | AAAGCCGCTCAACTAC | TGCTTTGAATGCGTCCC | 149 |
| | | ATGG (SEQ ID NO: 15) | AGAG (SEQ ID NO: 16) | |
| | CYP2D6 | GCTTCTCCGTCTCCAC | TGCCCTTCTGCCCATCA | 130 |
| | | CTTG (SEQ ID NO: 17) | C (SEQ ID NO: 18) | |
| | GSTT1 | CATCCTGCTCTACCTGA | GCTTCTCCGCAGAGTCG | 121 |
| | | CGC (SEQ ID NO: 19) | TG (SEQ ID NO: 20) | |
| | GSTM1 | ATCACCCAGAGCAACG | ACAGCCCAGCCAAGGA | 111 |
| | | C (SEQ ID NO: 21) | (SEQ ID NO: 22) | |
| | UGT2B17 | GAATTCATCATGATCAA | ACAGGCAACATTTTGTGA | 201 |
| | | CCG (SEQ ID NO: 23) | TC (SEQ ID NO: 24) | |
| | SULT1A1 | TAATCCGAGCCTCCAC | GAAGTCCACGGTCTCCT | 110 |
| | | T (SEQ ID NO: 25) | C (SEQ ID NO: 26) | |
| Long-PCR | CYP2D6 | TCCTCCTTCCACCTGCT | CCCTGACACTCCTTCTTG | 2347 |
| | | CACTCC (SEQ ID NO: 27) | CCTCC (SEQ ID NO: 28) | |
| | GSTT1 | AGACCGAACAGAGAAA | GAACTGGAATAGCAGGA | 2470 |
| | | GGTGAGC (SEQ ID NO: 29) | AGGCAA (SEQ ID NO: 30) | |
| | GSTM1 | CCTCAACCACATGTCC | TCTGTCCTCACTGACCTC | 2503 |
| | | CCTCTC (SEQ ID NO: 31) | CCAA (SEQ ID NO: 32) | |
| | UGT2B17 | TTTCTGTTCCCTCCTTC | CAAGTCCTTTTTTTTGAC | 2473 |
| | | CTATG (SEQ ID NO: 33) | CTCC (SEQ ID NO: 34) | |
| | SULT1A1 | AAGGTCACAGCATCCT | GCCAGAAATACTATCATC | 2134 |
| | | GAGAACAT (SEQ ID NO: 35) | CCCATC (SEQ ID NO: 36) | |
| PCR-RFLP | CYP2D6 | GCCTTCGCCAACCACT | AAATCCTGCTCTTCCGA | 334 |
| | | CCG (SEQ ID NO: 37) | GGC (SEQ ID NO: 38) | |
| | SULT1A1 | GTTGGCTCTGCAGGGT | CCCAAACCCCCTGCTGG | 333 |
| | | TTCTAGGA (SEQ ID NO: | CCAGCACCC (SEQ ID | |
| | | 39) | NO: 40) | |
| | TP53 | ATCTACAGTCCCCCTTG | GCAACTGACCGTGCAAG | 296 |
| | | CCG (SEQ ID NO: 41) | TCA (SEQ ID NO: 42) | |

qPCR reactions were conducted using the SYBR Green I PCR KIT (Qiagen) on a Rotor Gene Q (Qiagen). Each reaction was run in duplicate and contained 3 μL of DNA template (60 ng) along with 200 nM (LINE1 and GSTT1) or 400 nM (CYP2D6, GSTM1, UGT2B17 and SULT1A1) of primers and 1× Power SYBR® Green PCR Master Mix in a final reaction volume of 15 μL. Cycling parameters were: 95° C. for 5 min to activate DNA polymerase, then 40 cycles of 95° C. for 5 s and 60° C. for 10 s, with a final recording step of 75° C. for 10 s. Melting curves were performed after each run to ensure that only a single product was amplified. Data were analyzed using Rotor Gene Q Series Software (Qiagen).

In order to transform the relative values obtained after qPCR into absolute values, the number of copies of each gene was measured by Fluorescence In Situ Hybridization (FISH) in the control DNA used as calibrator. First, long PCR was performed for each gene to generate FISH probes. Primers were designed with the Primer Premier Biosoft software (Table 2) and DNA from a healthy individual was used as template. The PCR was performed in a total volume of 20 μL comprising 100 ng of DNA, 1×PCR buffer, 1.5 mM MgCl2, 0.2 mM dNTPs, 0.2 μM of each primer, and 0.5 U Taq polymerase (Promega Corporation, Madison, Wis.). The PCR conditions were: initial denaturation at 94° C. for 2 min, followed by 35 cycles of denaturation for 15 s at 94° C., annealing for 40 s at 57° C. (UGT2B17), 59° C. (SULT1A1), 64° C. (CYP2D6) or 65° C. (GSTT1 and GSTM1) and extension for 2 min 20 s at 68° C., with a final extension step at 68° C. for 5 min.

The amplified fragments were labeled by standard nick translation procedures using the Nick translation kit (Roche, Basel, Switzerland). Briefly, a 50-μL solution containing 1 μg of long PCR product, 1× Nick Translation Buffer, 0.02 mM dNTP mix (without dTTP), 0.01 mM of dTTP, 0.01 mM Spectrum Orange-dUTP, and 10 μL of Nick Translation Enzyme was run in a thermal cycler at 15° C. for 16 h and at 70° C. for 10 min. Finally, these probes were hybridized to nuclei spreads from the qPCR control individual, as follows. DNA was ethanol precipitated along with 12.5 ng of salmon sperm DNA and redissolved in 50 μL of TE. Then, 20 μL of labeled DNA probe and 10 ng of Cot1 competitor DNA (Thermo Scientific, Rockford, Ill.) were mixed and dried at 60° C., and resuspended in 5 μL of 1× hybridization buffer (50% formamide, 2×SSC (Saline Sodium Citrate) and 10% dextran sulfate). Labeled probes were denatured at 73° C. for 8 min, added to the denatured spreads (2 min at 73° C. in 70% formamide) and then allowed to hybridize overnight at 37° C. The slides were washed in a series of buffers: 0.7×SSC/0.3% NP-40 at 73° C. for 1 min, and 2×SSC/0.1% NP-40 at room temperature for 1 min. Interphase nuclei were analyzed in a Zeiss Axio Imager.Z2 microscope (Carl Zeiss Meditec A G, Jena, Germany) coupled to a Metafer® Slide Scanning System v3.10.2 (MetaSystems, Altlussheim, Germany).

FISH experiments showed that control DNA had one copy of GSTT1, GSTM1, and UGT2B17 genes and two copies of CYP2D6 and SULT1A1 genes. After CNV genotyping of CYP2D6, GSTT1, GSTM1, UGT2B17 and SULT1A1, the relative qPCR values obtained (sample vs. control DNA) were transformed into absolute gene copy numbers by dividing them by the number of copies of each gene in the control DNA (obtained by FISH).

SNP Genotyping by PCR-Restriction Fragment Length Polymorphism (PCR-RFLP)

The samples were genotyped for the most common SNPs in CYP2D6 (c.1846G>A, causing an splicing defect in protein), SULT1A1 (c.638G>A, p.Arg213His) and TP53 (c.215G>C, p.Arg72Pro) by PCR-RFLP method. PCR was performed in a 20-μL reaction volume containing 20 ng of genomic DNA (CYP2D6 and SULT1A1) or 100 ng (TP53), 1×PCR buffer, 1.5 mM MgCl₂, 200 μM dNTPs, 0.2 μM of each primer (Table 2), and 0.5 U Taq polymerase (Promega Corporation). Cycling conditions were: denaturation at 94° C. for 5 min followed by 35 cycles at 94° C. for 30 s, 58° C. (TP53) or 69° C. (CYP2D6 and SULT1A1) for 20 s, and 72° C. for 20 s, and a final extension step at 72° C. for 2 min. The PCR product (10 μL) was digested with BstNI (CYP2D6), HaeII (SULT1A1) or BstUI (TP53) digestion enzymes (10 U/μL; Thermo Scientific) according to manufacturer's specifications and digestion products were subjected to electrophoresis on a 3% agarose gel. The results obtained showed that for CYP2D6 digestion, the wild-type homozygous genotype (G/G) yielded two bands (230 and 104 bp), the heterozygous genotype (G/A) yielded three bands (334, 230, and 104 bp) and the mutant genotype (NA) yielded one band (334 bp). As for SULT1A1 digestion, the wild-type homozygous genotype (G/G) yielded two bands (168 and 165 bp), the heterozygous genotype (G/A) yielded three bands (333, 168 and 165 bp), and the mutant genotype (NA) yielded one band (333 bp). Regarding TP53 digestion, the Arg homozygous genotype (G/G) was cleaved by BstUI and yielded two small fragments (160 and 119 bp), the Pro homozygous genotype (C/C) was not cleaved and yielded a single 279 bp band, and the heterozygote showed three bands (279, 160, and 119 bp).

Statistical Analysis

First, potential prognostic variables, such as clinical parameters, were correlated with treatment efficacy variables (early response, MRD, and relapse). Chi-square and Fisher exact tests were used for sex, leukemia type, cytogenetic risk group and treatment protocol, whereas Mann-Whitney test was used for age and WBC count.

Second, association analysis between polymorphism genotypes and treatment efficacy variables was performed using R 3.3.1 package (http://www.r-project.org/). In particular, SNPassoc library (Gonzalez J R, et al. Bioinformatics. 2007; 23(5):644-645) was used to test SNP genotypes and CNVassoc library (Subirana I, et al. BMC Med. Genomics. 2011; 4(1):47) for CNV genotypes. SNPassoc implements logistic regression methods under five different genetic models (codominant, dominant, recessive, overdominant and log-additive). In addition, SNPassoc explores Hardy-Weinberg equilibrium (HWE) in each SNP using a chi-square test and it can perform an epistasis analysis (SNP-SNP interaction) between all pairs of SNPs. CNVassoc is a latent class model that incorporates uncertainty when copy number status is inferred. For example, copy number status can be assigned by defining a set of cutoff points in the signal distribution. It also includes functions for analyzing association under various inheritance models (additive, multiplicative, dominant or recessive). Both packages allow to adjust for covariates.

All possible combination of CNVs (e.g. null genotype in both GSTT1 and GSTM1 vs. other genotypes) and different combinations of SNP and CNV genotypes (Table 3) were also tested for associations with the three therapeutic outcome variables by chi-square test or Fisher exact test.

TABLE 3
List of combinations of CNV genotypes and SNPs
genotypes, which were tested for association with
therapeutic outcome variables and survival.

| CNV | | |
|---|---|---|
| (number of copies) | AND/OR | SNP* |
| SULT1A1 >2 | AND | SULT1A1 GG |
| SULT1A1 >2 | OR | SULT1A1 GG |
| SULT1A1 >2 | AND | SULT1A1 GG & GA |
| SULT1A1 >2 | OR | SULT1A1 GG & GA |
| CYP2D6 >2 | AND | CYP2D6 GG |
| CYP2D6 >2 | OR | CYP2D6 GG |
| CYP2D6 >2 | AND | CYP2D6 GG & GA |
| CYP2D6 >2 | OR | CYP2D6 GG & GA |
| GSTM1 null | AND | TP53 GG (Arg/Arg) |
| GSTM1 ≥1 | AND | TP53 CC (Pro/Pro) & |
| | | CG (Pro/Arg) |

*G allele is conferring high expression of SULT1A1 and CYP2D6.

Finally, Kaplan-Meier curves were generated for graphical representation of overall survival (OS) and event-free survival (EFS) for all clinico-biological parameters and for all polymorphism genotypes. OS is defined as time from diagnosis to death from any cause or last follow-up. EFS is the time to relapse, death from any cause or last follow-up. Mean follow-up was 83±42 months (range 6-204). Survival curves were compared with the log-rank test. Gene copy number of CYP2D6, GSTT1, GSTM1, UGT2B17, and SULT1A1 was categorized according to the cutoff points from the CNVassoc analysis. Every genotype was analyzed separately and moreover, in case of CNVs, null genotypes were tested vs. non-null genotypes, and in case of SNPs, heterozygotes were grouped together with either homozygote genotype.

Additionally, the same combinations of SNP and CNV genotypes above-mentioned were also tested in survival analysis. Moreover, the Cox proportional hazards regression model was used to adjust the effect of genotype status by potential prognostic features, such as clinico-biological parameters (sex, age, leukemia type, WBC count, cytogenetic risk group and treatment protocol).

All statistical analyses were performed using SPSS v22.0 software, unless otherwise indicated.

In Vitro Analysis of Cell Viability

Jurkat cell line, which is derived from a human lymphoblastic T-cell leukemia, was used (American Type Culture Collection, ATCC). Jurkat p53Pro and p53Arg cells were obtained by transfecting vectors with the two TP53 variants at codon 72, either Pro or Arg, into Jurkat cells, which are TP53 null. Vectors were kindly donated by Dr. Lawrence Banks (International Centre for Genetic Engineering and Biotechnology, Trieste, Italy) (Thomas M., et al. Mol Cell Biol. 1999 February; 19(2):1092-100). Inserts from these plasmids were subcloned into the pLNCX2 retroviral vector (Clontech laboratories, Palo Alto, Calif.) to construct pLNCX2-p53Arg and pLNCX2-p53Pro (HindIII and NotI sites) following standard methods.

To generate cell lines that stably express p53Arg or p53Pro, retroviral production and infection was carried out. First, pLNCX2-p53Arg and pLNCX2-p53Pro constructs were transiently transfected into the Phoenix packaging cell line with jetPEI® (Polyplus, Illkirch, France) according to the manufacturer's protocol. For retroviral infection, Jurkat cells were incubated in the presence of the retrovirus-containing supernatant and 4 μg/mL polybrene (Sigma-Aldrich, Taufkirchen, Germany) for 24 h. Infection was repeated the next day. Twenty-four hours after the second infection, medium supplemented with G418 (1 mg/mL, Sigma-Aldrich) was added, and cells underwent selection for 3 days to eliminate uninfected cells. Standard Western blot analysis was carried out to confirm p53 expression.

Jurkat p53Pro and Jurkat p53Arg cells were electroporated with GSTM1 siRNA (Santa Cruz Biotechnology, Dallas, Tex.) at a final concentration of 600 nM and then cultured for 72 h in RPMI-1640 medium containing L-glutamine (Invitrogen, Carlsbad, Calif.) supplemented with 15% fetal bovine serum and 1% penicillin-streptomycin. At this point, we had four cell lines: Jurkat p53Pro GSTM1 non-null, Jurkat p53Pro GSTM1 null, Jurkat p53Arg GSTM1 non-null, and Jurkat p53Arg GSTM1 null. Then, dexamethasone was added at varying concentrations with a final volume of 100 μL (1, 10, 100, 200 and 400 μM); cell viability was measured 72 hours later using the MTT assay. Briefly, 10 μL of MTT (5.5 mg/mL) was added to each well. After 2 h of incubation, 100 mL of solubilization solution was added to dissolve the crystals. The plate was allowed to stand 1 h incubation and the absorbance at 550 nm was recorded. Negative control was set as 100% of cell survival. Six replicates of each drug concentration were tested and the experiments were repeated once. Differences in percentage of viable cells at each concentration point were evaluated by Kruskal-Wallis test (comparing the four cell lines) or Mann-Whitney test (comparing Jurkat p53Arg GSTM1 null vs. Jurkat p53Pro GSTM1 non-null) with SPSS v22.0 software.

### II. Results

A total of 173 Caucasian children with ALL were enrolled in this study, with a mean age of 6.5±4 years. Samples were genotyped for polymorphisms on gene copy number and/or SNPs in six genes encoding several xenobiotic metabolizing enzymes (CYP2D6, GSTT1, GSTM1, UGT2B17 and SULT1A1) and an apoptotic protein (TP53). Genotype distributions of gene copy numbers for each gene are shown in Table 4.

TABLE 4
Genotype distribution for CNVs of CYP2D6, GSTT1, GSTM1,
UGT2B17, and SULT1A1 in patients with childhood ALL.

| | | | | | Samples |
|---|---|---|---|---|---|
| | | | | | success- |
| | 0 copies | | | | fully |
| Gene | (null) | 1 copy | 2 copies | ≥3 copies | genotyped |
| CYP2D6 | 2 (1.2%) | 25 (14.5%) | 127 (73.8%) | 18 (10.5%) | 172 |
| GSTT1 | 29 (16.8%) | 87 (50.3%) | 51 (29.5%) | 6 (3.4%) | 173 |
| GSTM1 | 89 (51.4%) | 70 (40.5%) | 9 (5.2%) | 5 (2.9%) | 173 |
| UGT2B17 | 20 (11.6%) | 75 (43.6%) | 67 (39%) | 10 (5.8%) | 172 |
| SULT1A1 | 0 (0%) | 9 (5.2%) | 120 (69.8%) | 43 (25%) | 172 |

Samples were also genotyped for the most common SNP in CYP2D6, SULT1A1 and TP53 and the genotype distribution is listed on Table 5. None of the SNP genotypes showed significant deviations from HWE, except CYP2D6 (p=0.03).

TABLE 5
Genotype distribution for SNPs at CYP2D6, SULT1A1
and TP53 in patients with childhood ALL.

| | | | | | Samples |
|---|---|---|---|---|---|
| | | Homoz. | Homoz. | Homoz. | successfully |
| SNP | Heteroz. | G | C | A | genotyped |
| CYP2D6 | 44 | 106 | — | 12 | 162 |
| (c.1846G > A) | (27.2%) | (65.4%) | | (7.4%) | |
| SULT1A1 | 76 | 78 | — | 15 | 169 |
| (c.638G > A) | (45%) | (46.1%) | | (8.9%) | |
| TP53 | 61 | 18 | 70 | — | 149 |
| (c.215G > C) | (40.9%) | (12.1%) | (47%) | | |

Heteroz.: Heterozygous; Homoz. G: Homozygous G; Homoz. C: Homozygous C; Homoz. A: Homozygous A.

Statistical analysis revealed no significant association between the patients' clinical parameters (sex, age, leukemia type, WBC count, cytogenetic risk group and treatment protocol) and treatment efficacy variables (early response, MRD and relapse) (p>0.05 in all cases). Kaplan-Meier survival curves showed significant differences for age (OS, p=0.002 and EFS, p=0.080) (FIG. 1), for cytogenetic risk group (OS, p=0.004 and EFS, p=0.085) (FIG. 2) and for MRD (OS, p=0.024 and EFS, p=0.028) (FIG. 3). Moreover, there was a trend to worse survival depending on the leukemia type (OS, p=0.168) (FIG. 4) and early response (OS, p=0.085 and EFS, p=0.088) (FIG. 5). Noteworthy, none of the patients died from bone marrow transplant complications.

To determine whether the CNV and SNP genotypes were associated with the treatment efficacy variables, CNVassoc and SNPassoc libraries were applied, respectively. No associations were found with any of the genetic models tested in any of the genes. Then, the impact of the CNVs and/or SNPs on patient survival was assessed by estimating OS and EFS probabilities for patients with or without the variant genotypes. The only significant predictor of survival was the GSTM1 CNV genotype. Kaplan-Meier survival analysis revealed worse survival in patients with 1, 2 or ≥3 copies of GSTM1 both when genotypes were analyzed separately (OS, p=0.013, FIG. 6A; EFS, p=0.047, FIG. 6B) and when all non-null genotypes were grouped together (OS, 86% vs. 99%, p=0.002, FIG. 6C; EFS, 80% vs. 94%, p=0.005, FIG. 6D). These differences were also observed when the effect was adjusted by clinico-biological factors in the Cox model for OS (hazard ratio [HR]=16.514; 95% confidence interval [Cl], 2.128-128.176; p=0.007) and EFS (HR=3.800; 95% Cl, 1.402-10.303; p=0.009). Therefore, GSTM1 is a genetic marker for childhood ALL survival since non-null genotype of GSTM1 was associated with poor prognosis, even after multivariate analysis. It made no difference whether one, two, or even more than two copies were present, as can be observed in survival curves (FIG. 6). Therefore, it seems that non-null genotype is the determinant factor to shift the balance from good to poor prognosis, regardless of the number of copies.

Additionally, survival curves for patients grouped according to TP53 gene polymorphism at codon 72 clearly separated individuals with the Arg/Arg genotype (Arg related to higher apoptosis efficiency) from those carrying at least one Pro variant, the latter having worse survival (FIG. 7) (p=0.208 for OS and p=0.110 for EFS)

The inventors further analyzed interactions among SNPs and among CNVs. Epistasis analysis (gene-gene interaction) performed with SNPassoc library did not show any significant association. Moreover, all possible combinations of CNV genotypes were analyzed but they were not statistically significantly associated with early response, MRD, relapse or survival. Specifically, double null GSTM1 and GSTT1 genotype was not a significant risk factor for any of the treatment outcome variables.

Then, it was tested whether the combination of a CNV genotype in one gene with a SNP genotype in the same gene having the same theoretical effect, such as increase of gene expression, was associated with treatment efficacy or survival. This analysis was performed for CYP2D6 and SULT1A1. There were no significant differences when testing patients having both >2 copies of the gene and the allele of the SNP conferring high gene expression (homozygous or homozygous+heterozygous) versus the rest of the patients. Neither did they observe differences when they grouped together patients having either >2 copies of the gene or the allele of the SNP conferring high gene expression, compared with the rest of patients.

The inventors next analyzed the importance of combining GSTM1 CNV genotype and the TP53 Arg72Pro SNP genotype, being both genes implicated in apoptosis processes. When combining GSTM1 and TP53 genotypes, the data indicated that they can predict survival with higher significance than GSTM1 alone (OS, p=0.0005 vs. p=0.002; EFS, p=0.003 vs. p=0.005). Children with GSTM1 non-null genotype and TP53 Pro/Pro or Pro/Arg genotype were more likely to have shorter survival than children with other genotypes. Patients having both non-null genotype of GSTM1 and TP53 Pro/Pro or Pro/Arg genotype had higher risk of relapse than other patients (p=0.043), but no association was found with early response or MRD. Other combinations tested did not show any significant association with therapy outcome variables.

In relation to survival, again patients having >1 copies of GSTM1 and TP53 Pro/Pro or Pro/Arg (n=62/149 patients with both genes successfully genotyped) had significantly poorer OS (82% vs. 99%, p=0.0005) and EFS (77% vs. 94%, p=0.003) (FIG. 8). These differences remained highly significant after applying multivariate Cox regression, either in OS (HR=19.098; 95% Cl, 2.444-149.212; p=0.005) or EFS (HR=4.135; 95% Cl, 1.488-11.487; p=0.006).

After observing that GSTM1 CNV and TP53 Arg72Pro SNP had an effect on OS and EFS of patients with ALL, the inventors performed an in vitro analysis to measure the effect of these polymorphisms on the response to a chemotherapy agent, dexametasone.

In this sense, synthetic glucocorticoids, such as dexamethasone and prednisone, are the keystone in the treatment of ALL in children due to their ability to directly induce extensive apoptosis in ALL cells, through activation of p38-MAPK and Bim.

Response to these agents is probably the most important prognostic factor for outcome in this disease. Moreover, it is known that the in vitro response of leukemic cells to glucocorticoids is highly predictive of ALL outcome.

Jurkat p53Pro and Jurkat p53Arg cells were electroporated with a siRNA of GSTM1 and the effect of GSTM1 expression and TP53 variant on cellular dexamethasone sensitivity was assayed (FIG. 9). Jurkat p53Arg GSTM1 null cells exhibited the highest sensitivity to dexamethasone and Jurkat p53Pro GSTM1 non-null the lowest, confirming the results observed in patients. Differences between these two cell lines were statistically significant at all concentrations tested (p=0.006 for 1 and 10 μM, and p=0.004 for 100, 200 and 400 μM). The other cell lines presented an intermediate sensitivity.

These data suggest that GSTM1 and TP53 polymorphisms are a major contribution for differences in cellular response to conventional chemotherapy such as glucocorticoids in childhood ALL.

1. (canceled)

2. (canceled)

3. (canceled)

4. A method for selecting a patient suffering from acute lymphoblastic leukaemia (ALL) for a conventional chemotherapy and treating said patient comprising
determining in a sample from said patient the polymorphisms selected from the copy number of GSTM1 gene comprising the SEQ ID NO: 5 and the rs1042522 comprising the SEQ ID NO: 11, wherein the absence of the GSTM1 gene and the presence of Arg/Arg genotype in the rs1042522 is indicative of the patient is selected for the conventional chemotherapy,
and selecting the patient for conventional chemotherapy, or administering the conventional chemotherapy to said patient.

5. A method according to claim 4 wherein the conventional chemotherapy is selected from the group consisting of glucocorticoids, vincristine, daunorubicin, L-asparaginase, methotrexate, cytarabine, mercaptopurine, cyclophosphamide, doxorubicin, tioguanine, vindesine, ifosfamide, etoposide, thioguanine, mercaptopurine or any combinations thereof.

6. A method according to claim 5 wherein the glucocorticoids are selected from the list consisting of prednisone, prednisolone, hydrocortisone, dexamethasone or any combinations thereof.

7. A method according to claim 4 wherein the patient is a child.

8. A method according to claim 4 wherein the biological sample is selected from biopsy sample, tissue, cell or fluid sample.

9. A method according to claim 8 wherein the fluid sample is selected from the list consisting of blood, peripheral blood, serum, plasma and saliva.

10. A method according to claim 4 wherein the determination of the polymorphisms is performed out by quantitative polymerase chain reaction (QPCR), Real-Time PCR (RT-qPCR), Retro-Transcriptase PCR (RT-PCR), long-range PCR, Restriction Fragment Length Polymorphism-PCR (PCR-RFLP), a DNA array, a, RNA array or nucleotide hybridization technique.

11. (canceled)

12. A kit or device for in vitro predicting the clinical outcome of a patient suffering from acute lymphoblastic leukaemia (ALL), for in vitro predicting the efficacy of a conventional chemotherapy in a patient suffering from ALL, or for in vitro selecting a patient suffering from ALL for a conventional chemotherapy, said kit comprising reagents for analysing a biological sample from said subject for the presence or absence of one or more polymorphisms selected from the copy number of GSTM1 gene comprising the SEQ ID NO: 5 and the rs1042522 comprising the SEQ ID NO: 11, wherein said reagents are selected from the group consisting of probes, primers, and/or oligonucleotides specific for each of said polymorphisms.

13. A kit or device according to claim 12 wherein the probes, primers and/or oligonucleotides are selected from the list consisting of SEQ ID NO: 15 to SEQ ID NO: 42, or any combinations thereof.

14. (canceled)

15. A method accordingly to claim 4, comprising administering the conventional therapy to the patient.

16. A method of treating a patient suffering from acute lymphoblastic leukaemia (ALL), comprising:
administering a conventional chemotherapy to the patient, wherein the patient has been determined to have a favourable clinical outcome in response to conventional chemotherapy by a method comprising:
determining in a sample from said patient the polymorphisms selected from the copy number of GSTM1 gene comprising the SEQ ID NO: 5 and the rs1042522 comprising the SEQ ID NO: 11, and
determining that said patient has an absence of the GSTM1 gene and the presence of Arg/Arg genotype in the rs1042522.

17. A method according to claim 16 wherein the conventional chemotherapy is selected from the group consisting of glucocorticoids, vincristine, daunorubicin, L-asparaginase, methotrexate, cytarabine, mercaptopurine, cyclophosphamide, doxorubicin, tioguanine, vindesine, ifosfamide, etoposide, thioguanine, mercaptopurine or any combinations thereof.

18. A method according to claim 17 wherein the glucocorticoids are selected from the list consisting of prednisone, prednisolone, hydrocortisone, dexamethasone or any combinations thereof.

19. A method according to claim 16 wherein the patient is a child.

20. A method according to claim 16 wherein the biological sample is selected from biopsy sample, tissue, cell or fluid sample.

21. A method according to claim 20 wherein the fluid sample is selected from the list consisting of blood, peripheral blood, serum, plasma and saliva.

22. A method according to claim 16 wherein the determination of the polymorphisms is performed out by quantitative polymerase chain reaction (QPCR), Real-Time PCR (RT-qPCR), Retro-Transcriptase PCR (RT-PCR), long-range PCR, Restriction Fragment Length Polymorphism-PCR (PCR-RFLP), a DNA array, a, RNA array or nucleotide hybridization technique.
